SEQUENCE LISTING (75 sequences)

SEQ ID NO: 1 - protein, 897 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 2 - protein, 14 aa, Homo sapiens
NGPYLGPPHSRSLP

SEQ ID NO: 3 - protein, 13 aa, Homo sapiens
NVHYRTPKTHTMP

SEQ ID NO: 4 - protein, 15 aa, Homo sapiens
RYFTQKEETESGSGP

SEQ ID NO: 5 - protein, 22 aa, Homo sapiens
NKKYELQDRDVCEPRYRSVSEP

SEQ ID NO: 6 - protein, 13 aa, Homo sapiens
NPTYSVMRSHSYP

SEQ ID NO: 7 - protein, 24 aa, Homo sapiens
NIFYLIRKSGSFPMPELKLSISFP

SEQ ID NO: 8 - protein, 19 aa, Homo sapiens
NEEYLDLSQPLEQYSPSYP

SEQ ID NO: 9 - protein, 19 aa, Homo sapiens
NQEYLDLSMPLDQYSPSFP

SEQ ID NO: 10 - protein, 16 aa, Homo sapiens
NATYKVDVIQRTRSKP

SEQ ID NO: 11 - protein, 11 aa, Homo sapiens
NPEYHSASSGP

SEQ ID NO: 12 - protein, 10 aa, Homo sapiens
NPDYWNHSLP

SEQ ID NO: 13 - protein, 23 aa, Homo sapiens
NPSYSSNPFVNYNKTSICSKSNP

SEQ ID NO: 14 - protein, 11 aa, Homo sapiens
NTLYFNSQSSP

SEQ ID NO: 15 - protein, 24 aa, Homo sapiens
NPVYQKTTEDEVHICHNQDGYSYP

SEQ ID NO: 16 - protein, 24 aa, Rattus sp.
NPVYLKTTEEDLSIDIGRHSASVG

SEQ ID NO: 17 - protein, 38 aa, Homo sapiens
NPTYKMYEGGEPDDVGGLLDADFALDPDKPTNFTNPVY

SEQ ID NO: 18 - protein, 12 aa, Homo sapiens
NPIYKSAVTTVV

SEQ ID NO: 19 - protein, 12 aa, Homo sapiens
NPLYKSAITTTV

SEQ ID NO: 20 - protein, 12 aa, Homo sapiens
NPLYKEATSTFT

SEQ ID NO: 21 - protein, 12 aa, Homo sapiens
NPLYRKPISTHT

SEQ ID NO: 22 - protein, 12 aa, Homo sapiens
NPLYRGSTSTFK

SEQ ID NO: 23 - protein, 12 aa, Homo sapiens
PGHYLRCDSTQP

SEQ ID NO: 24 - protein, 17 aa, Homo sapiens
VQTYVLQGDPRAVSTQP

SEQ ID NO: 25 - protein, 14 aa, Homo sapiens
QVLYGQLLGSPTSP

SEQ ID NO: 26 - protein, 22 aa, Homo sapiens
HSGYRHQVPSVQVFSRSESTQP

SEQ ID NO: 27 - protein, 17 aa, Homo sapiens
WKMYEVYDAKSKSVSLP

SEQ ID NO: 28 - protein, 16 aa, Homo sapiens
KIPYFHAGGSKCSTWP

SEQ ID NO: 29 - protein, 19 aa, Homo sapiens
ELDYCLKGLKLPSRTWSPP

SEQ ID NO: 30 - protein, 15 aa, Homo sapiens
SGDYMPMSPKSVSAP

SEQ ID NO: 31 - protein, 38 aa, Homo sapiens
SFYYSEENKLPEPEELDLEPENMESVPLDPSASSSSLP

SEQ ID NO: 32 - protein, 22 aa, Homo sapiens
EEIYIIMQSCWAFDSRKRPSFP

SEQ ID NO: 33 - protein, 14 aa, Homo sapiens
ISQYLQNSKRKSRP

SEQ ID NO: 34 - protein, 11 aa, Homo sapiens
GTAYGLSRSQP

SEQ ID NO: 35 - protein, 15 aa, Homo sapiens
YLPQEDWAPTSLTRP

SEQ ID NO: 36 - protein, 43 aa, Homo sapiens
  MOD_RES (38): p-Ser
LVAYIAFKRWNSCKQNKQGANSRPVNQTPPPEGEKLHSDSGIS

SEQ ID NO: 37 - protein, 13 aa, Homo sapiens
NVHYRTPTTHTMP

SEQ ID NO: 38 - protein, 10 aa, Homo sapiens
NKCYRGRSCP

SEQ ID NO: 39 - protein, 27 aa, Homo sapiens
NPNYTEFKFPQIKAHPWTKVFKSRTPP

SEQ ID NO: 40 - protein, 31 aa, Homo sapiens
NQKYMSFTSGDKSAHGYIAAHPSSTKTASEP

SEQ ID NO: 41 - protein, 32 aa, Homo sapiens
NRTYYLMDPSGNAHKWCRKIQEVWRQRYQSHP

SEQ ID NO: 42 - protein, 24 aa, Homo sapiens
NIFYLIRKSGSFPMPELKLSISFP

SEQ ID NO: 43 - protein, 13 aa, Homo sapiens
NGPYLGPPHSRSL

SEQ ID NO: 44 - protein, 9 aa, Homo sapiens
NPDYWNHSL

SEQ ID NO: 45 - protein, 18 aa, Homo sapiens
NQEYLDLSIPLDQYSPSF

SEQ ID NO: 46 - protein, 18 aa, Homo sapiens
NEEYLDLSQPLEQYSPSY

SEQ ID NO: 47 - protein, 15 aa, Homo sapiens
NATYKVDVIQRTRSK

SEQ ID NO: 48 - protein, 35 aa, Homo sapiens
NPTYKMYEGGEPDDVGGLLDADFALDPDKPTNFTN

SEQ ID NO: 49 - protein, 23 aa, Homo sapiens
NPVYQKTTEDEVHICHNQDGYSY

SEQ ID NO: 50 - protein, 23 aa, Homo sapiens
NPVYLKTTEEDLSIDIGRHSASV

SEQ ID NO: 51 - protein, 12 aa, Homo sapiens
NVHYRTPTTHTM

SEQ ID NO: 52 - protein, 9 aa, Homo sapiens
NKCYRGRSC

SEQ ID NO: 53 - protein, 26 aa, Homo sapiens
NPNYTEFKFPQIKAHPWTKVFKSRTP

SEQ ID NO: 54 - protein, 30 aa, Homo sapiens
NQKYMSFTSGDKSAHGYIAAHPSSTKTASE

SEQ ID NO: 55 - protein, 31 aa, Homo sapiens
NRTYYLMDPSGNAHKWCRKIQEVWRQRYQSH

SEQ ID NO: 56 - protein, 23 aa, Homo sapiens
NIFYLIRKSGSFPMPELKLSISF

SEQ ID NO: 57 - protein, 11 aa, Homo sapiens
NPIYKSAVTTV

SEQ ID NO: 58 - protein, 13 aa, Homo sapiens
NPLYKEATSTFTN

SEQ ID NO: 59 - protein, 11 aa, Homo sapiens
NPLYRGSTSTF

SEQ ID NO: 60 - protein, 12 aa, Homo sapiens
NPLYKSAITTTI

SEQ ID NO: 61 - protein, 47 aa, Homo sapiens
RYSSDPTGALTEDSIDDTFLPVPEYINQSVPKRPAGSVQNPVYNPEY

SEQ ID NO: 62 - protein, 26 aa, Homo sapiens
KTLSPGKNGVVKDVFTFGGAVENPEY

SEQ ID NO: 63 - protein, 10 aa, Homo sapiens
RTHSLPNDSY

SEQ ID NO: 64 - protein, 28 aa, Homo sapiens
SDGPYSNPYENSLIPAAEPLPPSYVACS

SEQ ID NO: 65 - protein, 7 aa, Homo sapiens
HFSTELD

SEQ ID NO: 66 - protein, 45 aa, Homo sapiens
NQGYFFFHLPDALEIEACQVYFTYDPYSEEDPDEGVAGAPTGSSP

SEQ ID NO: 67 - protein, 23 aa, Artificial Sequence
  Description of Artificial Sequence Synthetic peptide
KGGFDFNGPYLGPPHSRSLPDGG

SEQ ID NO: 68 - protein, 23 aa, Artificial Sequence
  Description of Artificial Sequence Synthetic peptide
  MOD_RES (10): p-Tyr
KGGFDFNGPYLGPPHSRSLPDGG

SEQ ID NO: 69 - protein, 23 aa, Artificial Sequence
  Description of Artificial Sequence Synthetic peptide
  MOD_RES (18): p-Ser
KGGFDFNGPYLGPPHSRSLPDGG

SEQ ID NO: 70 - protein, 23 aa, Artificial Sequence
  Description of Artificial Sequence Synthetic peptide
  MOD_RES (10): p-Tyr
  MOD_RES (18): p-Ser
KGGFDFNGPYLGPPHSRSLPDGG

SEQ ID NO: 71 - protein, 24 aa, Artificial Sequence
  Description of Artificial Sequence Synthetic peptide motif
  MOD_RES (2)..(3): variable amino acid
  MOD_RES (5)..(17): region may encompass between 1 and 13 variable residues
  MOD_RES (18): Arg, Lys, His or Gln
  MOD_RES (19)..(21): region may encompass between 2 and 3 variable residues; see specification as filed for detailed description of preferred embodiments
  MOD_RES (22): Ser or Thr
  MOD_RES (23): variable amino acid
NXXYXXXXXXXXXXXXXXXXXXXP

SEQ ID NO: 72 - protein, 24 aa, Artificial Sequence
  Description of Artificial Sequence Synthetic peptide motif
  MOD_RES (2)..(17): region may encompass between 1 and 16 variable residues
  MOD_RES (18): Arg, Lys, His or Gln
  MOD_RES (19)..(21): region may encompass between 2 and 3 variable residues; see specification as filed for detailed description of preferred embodiments
  MOD_RES (22): Ser or Thr
  MOD_RES (23): variable amino acid
YXXXXXXXXXXXXXXXXXXXXXXP

SEQ ID NO: 73 - protein, 42 aa, Artificial Sequence
  Description of Artificial Sequence Synthetic peptide motif
  MOD_RES (2)..(3): variable amino acid
  MOD_RES (4): p-Tyr
  MOD_RES (5)..(34): region may encompass between 1 and 30 variable residues
  MOD_RES (35): Arg, Lys, Gln or His
  MOD_RES (36)..(39): region may encompass between 1 and 4 variable residues
  MOD_RES (40): p-Ser or p-Thr
  MOD_RES (41): variable amino acid
NXXYXXXXXXXXXXXXXXXXXXXXXXXXXXXXXXXXXXXXXP

SEQ ID NO: 74 - protein, 24 aa, Artificial Sequence
  Description of Artificial Sequence Synthetic peptide motif
  MOD_RES (2)..(3): variable amino acid
  MOD_RES (4): p-Tyr
  MOD_RES (5)..(17): region may encompass between 1 and 13 variable residues
  MOD_RES (18): Arg, Lys, His or Gln
  MOD_RES (19)..(21): region may encompass between 2 and 3 variable residues; see specification as filed for detailed description of preferred embodiments
  MOD_RES (22): p-Ser or p-Thr
  MOD_RES (23): variable amino acid
NXXYXXXXXXXXXXXXXXXXXXXP

SEQ ID NO: 75 - protein, 42 aa, Artificial Sequence; identical to SEQ ID NO: 73
  Description of Artificial Sequence Synthetic peptide motif
  MOD_RES (2)..(3): variable amino acid
  MOD_RES (4): Tyr
  MOD_RES (5)..(34): region may encompass between 1 and 30 variable residues
  MOD_RES (35): Arg, Lys, Gln or His
  MOD_RES (36)..(39): region may encompass between 1 and 4 variable residues
  MOD_RES (40): Ser or Thr
  MOD_RES (41): variable amino acid

(19) United States
(12) Patent Application Publication
(10) Pub. No.: US 2007/0219117 A1
(43) Pub. Date: Sep. 20, 2007

(54) BIDENTATE MOTIF AND METHODS OF USE

(75) Inventors: Mark Guthridge, South Australia (AU); Hayley Ramshaw, South Australia (AU); Frank Stomski, South Australia (AU); Fernando Felquer, South Australia (AU); Angel Lopez, South Australia (AU)

Correspondence Address: DARBY & DARBY P.C., P.O. BOX 770, Church Street Station, New York, NY, 10008-0770, US

(73) Assignee: MEDVET SCIENCE PTY LTD, Adelaide (AU)

(21) Appl. No.: 10/595,562

(22) PCT Filed: Oct. 27, 2004
(86) PCT No.: PCT/AU04/01482

§ 371 Date: Sep. 19, 2006

(30) Foreign Application Priority Data
Oct. 27, 2003 (AU) 2003905931

(51) Int. Cl. C07K 2/00

(52) U.S. Cl. 514/2; 435/375; 435/4; 530/350

(57) ABSTRACT

The present invention relates to a novel bidentate motif that is composed of two adjacent residues of tyrosine and serine which have been found to be involved in the binding of crucial cytoplasmic proteins which are involved in cell signalling pathways. In some cases, the cytoplasmic proteins are ubiquitous proteins involved in cell signalling pathways that may include mitogenesis, transformation and survival.
The bidentate motif may have a sequence alignment

(SEQ ID NO: 71)

N-X-X-Y- (X) ₁₋₁₃-[R/K/H/Q]-[X/Ψ] ₂₋₃-S/T-X-P;

(SEQ ID NO: 72)

Y- (X) ₁₋₁₆-[R/K/H/Q]-[X/Ψ] ₂₋₃-S/T-X-P;
or

(SEQ ID NO: 73)

N-X-X-Y-[X]₁₋₃₀-[R/K/Q/H]-[X]₁₋₄-[S/T]-X-p

wherein X is any residue, Y is tyrosine, S/T is serine or threonine and Ψ is a hydrophobic residue or an equivalent thereof. Preferably the residues are Tyr577 and Ser585 of the common βc of the GM-CSF/IL-5/IL-3 receptor.

### FIELD OF INVENTION

The present invention relates to a bidentate motif and use of the motif in methods of regulating cellular activities. The invention also includes methods of diagnosis of conditions relating to these cellular activities.

### BACKGROUND

Although many cytokines such as IL-3, GM-CSF and IL-5 and growth factors such as PDGF and IGF-1 were initially discovered as mitogens by virtue of their ability to promote cell proliferation, many of these factors were later also found to be potent regulators of cell survival through their ability to suppress programmed cell death or apoptosis. These biological activities are regulated by the binding of the cytokine or growth factor to their cognate cell surface receptor which initiates an ordered series of signalling events that includes receptor dimerization, the activation of tyrosine kinases followed by the tyrosine phosphorylation of the receptor cytoplasmic tail, the binding of multiprotein signalling complexes to receptor phosphotyrosine residues via src-homology 2 (SH2) domains or phosphotyrosine-binding (PTB) domains and the activation of downstream signalling cascades that promote a cellular response. Although a large degree of redundancy has been encountered when attempting to ascribe specific signalling pathways to unique biological responses, one emerging concept is that cell proliferation and cell survival can be viewed as independent biological outcomes that are regulated by distinct, yet intimately entwined, signalling pathways.

Evidence that cell survival which is reliant on a variety of cellular activities, is regulated in an independent manner to cell proliferation comes from the identification of centralized signalling nodes that are professional regulators of cell survival in a variety of biological settings. One such example is the PI 3-kinase. The activation of PI 3-kinase has been observed in response to a wide range of cytokines and growth factors and leads to the generation of phosphatidyl inositol phosphates which in turn promote the activation of pleckstrin homology domain proteins such as the serine-threonine kinase, Akt (or protein kinase B). Akt is able to regulate cell survival through the phosphorylation of selected downstream targets that modulate key aspects of cell viability such as gene transcription (IκB kinase, FKHR1), protein translation (mTOR), cell metabolism (GSK3b), and apoptosis (BAD). A number of cytokines and growth factors that are known to be potent regulators of cell survival such as interleukin-3 (IL-3), nerve growth factor (NGF), platelet-derived growth factor (PDGF) and insulin-like growth factor-1 (IGF-1) have been shown to regulate cell viability through PI 3-kinase signalling.

Despite the almost bewildering array of reports examining the regulation of intracellular signalling cascades by cytokines and growth factors, understanding the molecular mechanisms by which cell surface receptors initiate intracellular signalling pathways that specifically lead to cell survival and how these differ to those involved in regulating cell proliferation is largely unknown. Although there are many examples where specific tyrosine phosphorylation sites in cell surface receptors have been shown to be important in regulating cell proliferation, the receptor motifs involved in initiating cell survival signalling have proved far more elusive. Although there are clear instances where receptor tyrosine residues can be directly attributed to regulation of cell survival, there are many examples where receptor tyrosine phosphorylation cannot solely account for at least some of the biological activities of cytokines and growth factors. These results are suggestive of the possibility that some cell surface receptors may employ alternate novel receptor motifs for the regulation of cell survival.

It has been unclear until now how the binding of proteins to their receptors can result in the specialised functions associated with these molecules and their receptors. The signalling events which lead to the specialised functions are unknown. However various cellular proteins are implicated in the cascade of events culminating in the biological functions associated with various molecules. There are many ubiquitous proteins involved in cell signalling pathways and any one or more may be involved in relaying signals switched on by proteins binding to their receptor.

The 14-3-3 family of proteins is one such protein, which consists of 7 different isoforms and is expressed ubiquitously from yeast to humans. The ability of 14-3-3 to bind to a number of motifs in a wide range of signalling molecules suggests that 14-3-3 proteins may participate in a number of cell signalling pathways that may include mitogenesis, transformation and survival. Although 14-3-3 has been shown to bind a number of signalling molecules, it has been more difficult to determine how or where 14-3-3 can regulate signalling events directly or indirectly, or whether 14-3-3 is implicated at all.

Accordingly, an object of the present invention is to overcome some of the problems of the prior art and to understand how proteins can express their biological activities and to use this information to manipulate cellular functions.

### SUMMARY OF THE INVENTION

In a first aspect of the present invention there is provided a bidentate motif capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

The present invention relates to a novel bidentate motif that is composed of two adjacent residues of tyrosine and serine which have been found to be involved in the binding of crucial cytoplasmic proteins which are involved in cell signalling pathways. In some cases, the cytoplasmic proteins are ubiquitous proteins involved in cell signalling pathways that may include mitogenesis, transformation and survival.

In a preferred embodiment, the present invention provides a bidentate motif capable of binding to at least one cytoplasmic protein, said bidentate motif comprising a tyrosine and a serine/threonine residue which are capable of interaction with cytoplasmic proteins, and wherein the residue and cytoplasmic protein can interact to activate cellular activity in the cell and wherein at least one of the tyrosine or serine residues will bind to the cytoplasmic protein.

In an even further preferred embodiment, the cytoplasmic proteins are Shc for tyrosine and 14-3-3 for serine such that the Shc interacts with the 14-3-3 which in turn activates a binding of a signalling molecule which then activates a cell signalling pathway. Accordingly, the present invention preferably provides a tyrosine/serine bidentate motif that is essential for cell survival and a convergence of phosphoserine and tyrosine signals through a novel Shc/14-3-3 axis.

In another aspect of the present invention, there is provided a bidentate motif capable of binding to a cytoplasmic protein comprising a tyrosine and a serine/threonine residue, said motif consisting of the following sequence alignment:
N-X-X-Y-(X)₁₋₁₃-[R/K/H/Q]-[X/Ψ]₂₋₃-S/T-X-P
wherein X is any residue, Y is tyrosine, S/T is serine or threonine and Ψ is a hydrophibic residue or an equivalent thereof.

In yet another aspect of the present invention, there is provided a bidentate motif of a receptor capable of binding to a cytoplasmic protein comprising a tyrosine and a serine/threonine residue, said motif consisting of the following sequence alignment:
Y-(X)₁₋₁₆-[R/K/H/Q]-[X/Ψ]₂₋₃-S/T-X-P
wherein X is any residue, Y is tyrosine, S/T is serine or threonine and Ψ is a hydrophibic residue or an equivalent thereof.

In yet another aspect of the present invention, there is provided a bidentate motif capable of binding to a cytoplasmic protein comprising a tyrosine and a serine/threonine residue, said motif consisting of the following sequence alignment:
N-X-X-Y-[X]₁₋₃₀-[R/K/Q/H]-[X]₁₋₄-[S/T]-X-p
wherein X is any residue, Y is phosphotyrosine, S/T is phosphoserine/phosphothreonine.

In another aspect of the present invention there is provided a method of modulating cellular activities in a cell, said method comprising
modifying phosphorylation of a Tyr and/or Ser residue of a bidentate motif capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

Preferably the residues are Tyr577 and Ser585 of the common βc of the GM-CSF/IL-5/IL-3 receptor.

In another aspect of the present invention, there is provided a method of activating cellular activities in a cell said method including:
inducing phosphorylation of a bidentate motif, a functional equivalent or analogue thereof capable of binding a cytoplasmic protein and activating cellular activities in a cell, said bidentate motif comprising a tyrosine and a serine/threonine which are capable of interaction with cytoplasmic proteins, and wherein the residue and cytoplasmic protein can interact to activate cellular activity in the cell.and
subjecting the bidentate motif to a cytoplasmic protein to bind to the Tyr and/or Ser.

In yet another aspect of the present invention there is provided a method of modulating cellular activities in a cell, said method including:
modifying phosphorylation of a bidentate motif, a functional equivalent or analogue thereof capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell;
subjecting the bidentate motif to a cytoplasmic protein which binds to the tyrosine and serine residue; and
activating a cell signalling pathway by interacting the bound cytoplasmic protein with a signalling molecule involved in the pathway.

In another preferred aspect of the present invention, there is provided a method of inhibiting cell survival, said method including inhibiting the binding of a cytoplasmic protein to a bidentate motif capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

In another aspect of the invention, there is provided a method of inhibiting cell activation, said method including inhibiting the binding of a cytoplasmic protein to a bidentate motif, a functional equivalent or analogue thereof capable of binding a cytoplasmic protein which activates cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

In another aspect, there is also provided a method of treating a cytokine mediated condition in a cell said method comprising:
regulating the activation of phosphorylation of a bidentate motif, a functional equivalent or analogue thereof capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

### FIGURES

FIG. 1 shows the panel of constructs used to examine the role of Ser585 and Tyr577 residues in the ability of GM-CSF to regulate primary haematopoietic cell function.

FIG. 2 shows the double motif encompassing Tyr577 and Ser585 is necessary and sufficient for GM-CSF-mediated stimulation of colony formation from hemopoietic cells.

FIG. 3 shows the double mutant Y577F/S585G, but not the single mutants or F8 mutants, abolishes GM-CSF-stimulated cell survival.

FIG. 4 shows the double mutant Y577F/S585G is not defective in STAT5, Akt, JAK2, or Erk activation.

FIG. 5 shows Tyrosine 179 on 14-3-3 is critical for its association with Shc in response to GM-CSF stimulation.

STL-EN cells expressing the GM-CSFR were transfected with plasmids expressing wild type and mutant forms of 14-3-3-myc. 24 hs after transfection the cells were starved for 18 hrs in medium with 0.5% FCS in the absence of cytokine. After starvation cells were stimulated with GM-CSF (50 ng/ml) at 0, 5 and 15 minutes. Cells were then lysed and Shc immunoprecipitated using the anti-Shc antibody. Immunoprecipitates were subjected to immunoblot analysis using the anti myc antibody, the shc antibody. Tyrosine phosphorylation of the βc in response to GM-CSF was examined using the anti-phosphotyrosine monoclonal antibody 4G10 (Pβc). Lysates were also immunoblotted using βc antibodies to demonstrate equal loading.

FIG. 6 shows Tyrosine 179 on 14-3-3 is critical for its association with PI 3-kinase in response to GM-CSF stimulation.

CTL-EN cells expressing the GM-CSFR were transfected with plasmids expressing wild type and mutant forms of 14-3-3-myc. 34 hrs after transfection the cells were starved for 18 hrs in RPMI medium containing 0.5% FCS in the absence of cytokine. After starvation cells were stimulated with GM-CSF (50 ng/ml) for up to 5 minutes. Cells were then lysed and 14-3-3-myc was immunoprecipitated using a myc antibody and PI 3-k activity of the immunoprecipitates was measured. Shown are ³²P-labeled phosphatidylinositols (PIP) and the origin. (A). Quantification of the intensity of the ³²P-labeled phosphatidylinositols (PIP) is shown in B.

FIG. 7 shows Tyrosine 179 of 14-3-3 is required for Akt but not Erk activation in response to GM-CSF.

A) CTL-EN cells expressing the GM-CSFR were transfected with plasmids expressing wild type and mutant forms of 14-3-3-myc and Akt-HA. 24 hs after transfection the cells were starved for 24 hs in medium with 0.5% FCS in the absence of cytokine. After starvation cells were stimulated with GM-CSF (50 ng/ml) at 0, 5, 15 and 30 minutes. Cells were then lysed and cleared lysates were subjected to SDS-PAGE and immunoblotted sequentially using a cocktail of anti-phosphno-Akt-antibodies (Thr308 and Ser473), anti-HA antibody (12CA5) and anti-myc antibody (9E10). CTL-EN cells expressing the GM-CSFR were transfected with plasmids expressing wild type and mutant forms of 14-3-3-myc and Erk-HA. Cells were starved, stimulated with GM-CSF and lysed as described above. Lysates were subjected to SDS-PAGE and immunoblotted sequentially using an anti-phospho-Erk-antibody, anti-HA antibody (12CA5) and anti-myc antibody (9E10).

FIG. 8 shows cells from leukaemia patients exhibit phosphorylation of Ser585 (CML, Panel A) or both Ser585 and Tyr577 (AML, Panel B) of the βc chain. White cells are extracted from peripheral blood by percol gradient centrifugation, cultured for 2 hours in 10% FCS and then treated with GM-CSF. The βc-chain was immunoprecipitated with 1C1 and 8E4 (anti βc-chain), and probed with GMB (anti-phospho-serine 585), 4G10 (anti-phospho-tyrosine) and 1C1/8E4.

FIG. 9 shows the amino acid sequence of the common βc.

FIG. 10 shows Tyr577 and Ser585 constitute a distinct bidentate motif that is essential for the regulation of primary hemopoietic cell survival, proliferation and colony formation in response to GM-CSF. Fetal liver cells from βc −/− βIL-3 −/− double knockout mice were transduced with wt βc and mutant receptor constructs. A, B: Following transduction, cells were plated in either no factor (−), 3.3 μM hGM-CSF (+) or a positive control cytokine cocktail for 48 h. Cells were plated in either 0.1% or 10% FCS (A) or in 10% FCS (B). GM-CSF receptor-expressing cells (GMRα-PE positive) were then examined for cell viability (annexin V-FITC negative) by flow cytometry. C: Transduced GM-CSF receptor-expressing cells were purified by FACS and plated in either no factor, 3.3 μM hGM-CSF or the positive control cytokine cocktail for 24 h with a BrdU pulse for the last 4 h. Cells were then fixed and stained for BrdU incorporation and analysed by flow cytometry. D: Transduced cells were plated in soft agar in 3.3 μM GM-CSF or the positive control cytokine cocktail and cultured for 14 days. Colonies were then counted blind from triplicate plates. Results shown in A, B, C, D are representative of at least 2 experiments. Errors bars indicate standard deviations.

FIG. 11 shows Tyr577 and Ser585 function as a binary switch that couples to alternate signalling pathways and is deregulated in some leukemias. Pulldown experiments were performed using either non-phosphorylated (Y- or S-) or phosphorylated (Y-P or S-P) peptides encompassing Tyr577 (Y) and Ser585 (S) of βc (A). Lysates from HEK-293T cells or CTL-EN cells were subjected to pulldowns with the indicated peptides. Precipitates were then subjected to SDS-PAGE and immunoblotted with antibodies for p85, Shc or 14-3-3. Pulldown experiments were also performed on HEK-293T cell lysates and precipitates were subjected to SDS-PAGE and silver stained for the detection of 14-3-3 proteins. The ability of each peptide to precipitate purified recombinant 14-3-3ζ was also examined by immunoblot analysis using anti-14-3-3 antibodies. B, C, D: TF-1 cells were factor-deprived overnight in medium containing 0.5% FCS and then stimulated for 10 min with the indicated concentrations of GM-CSF (pM). Cells were then lysed and βc immunoprecipitated with the 1C1/8E4 mAbs. Immunoprecipitates were then subjected to SDS-PAGE and immunoblotted with anti-phospho-βcSer585, anti-phospho-βcTyr577, anti-14-3-3, anti-p85, anti-phospho-tyrosine (4G10) or anti-βc (1C1) antibodies (B, D). Cell lysates were also subjected to SDS-PAGE and immunoblotted with anti-phospho-JAK2, anti-phospho-STAT5 and anti-active ERK antibodies (D). The results in B were quantified by laser densitometry and the results are shown in C. Mononuclear cells from a normal donor (E) were purified and stimulated with the indicated concentrations of GM-CSF before lysis and βc immunoprecipitation with the 1C1/8E4 anti-βc mAbs. Immunopreciptates were then subjected to SDS-PAGE and immunoblotted as above. Results shown are representative of at least two experiments.

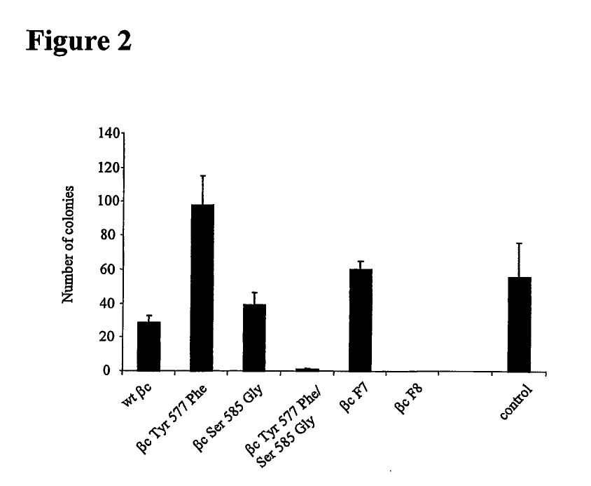

FIG. 12 shows the binary switch is deregulated in myeloid leukaemia. A. Mononuclear cells from a patient with AML were purified and stimulated with the indicated concentrations of GM-CSF before lysis and βc immunoprecipitation with the 1C1/8E4 anti-βc mAbs. Immunopreciptates were then subjected to SDS-PAGE and immunoblotted as in FIG. 2. B. Mononuclear cells (MNC) from either normal donors (▴) or patients with AML, CML, CMML and myelofibrosis (▪) were stimulated with GM-CSF and subjected to immunoblot analysis as above. Signals were quantified by laser densitometry scanning and the level of Ser585 phosphorylation in the absence of GM-CSF was plotted as a percentage of the maximum signal observed following GM-CSF stimulation.

FIG. 13 shows Concentration-dependent regulation of distinct biological functions by GM-CSF. TF-1 cells were plated out in the indicated concentrations of GM-CSF and cell viability or proliferation measured (A). Cells were cultured for 48 h and then stained with annexin V-FITC and cell survival (annexin V-FITC negative cells) was determined by flow cytometry (▪). For proliferation, cells were cultured for 24 h and pulsed with BrdU for 4 h. Cells were then fixed and stained with anti-BrdU and BrdU incorporation was determined by flow cytometry (♦). Primary human neutrophils were purified from peripheral blood and the ability of GM-CSF to promote cell survival or activation was examined (B). Neutrophils were cultured for 48 h in the indicated concentrations of GM-CSF and then stained with annexin V-FITC and viability was determined by flow cytometry (▪). For neutrophil activation, cells were stimulated with the indicated concentrations of GM-CSF for 75 min following which the cells were stained with anti-CD11 b-PE and the mean peak fluorescence measured by flow cytometry (σ). Results shown are representative of at least two experiments. Error bars indicate standard deviations.

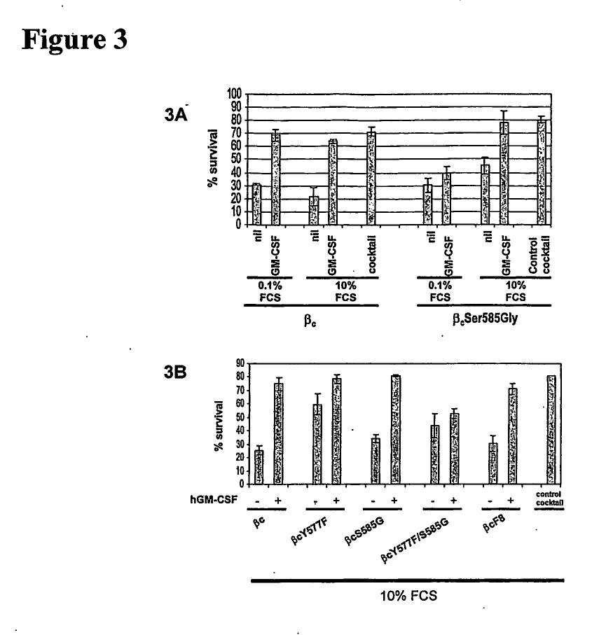

FIG. 14 shows Low concentrations of GM-CSF specifically promote cell survival via a pathway that involves PKA and PI 3-kinase and does not require phosphotyrosine signalling. Survival of neutrophils stimulated with GM-CSF in the presence of either forskolin, H89, LY294002 or vehicle (−)(A) or in the presence of 15 μM genestein, 15 μM AG490 or vehicle (−)(B) was determined as in FIG. 3B. PKA activity in TF-1 cells factor-deprived overnight and then stimulated with either 1 pM (σ) or 1000 pM (υ) GM-CSF or 25 μM forskolin (▪) for up to 30 min was determined as described in the Experimental Procedures (C). Shown are the counts per minute (cpm) incorporated into kemptide in duplicate samples. D. Phosphorylation of Ser473 of Akt in response to GM-CSF was measured in CTL-EN cells expressing the wt GM-CSF receptor. Cells were electroporated with a pCMV-AKT-HA construct (30 μg DNA/1×10⁷ cells) and 24 hours after electroporation were factor-deprived overnight in RPMI containing 0.5% FCS. Cells were then stimulated with either 1 pM or 1000 pM GM-CSF before lysis and subjected to immunoblot analysis with anti-phospho-Ser473 antibodies or anti-HA antibodies (12CA5). Results are representative of at least 3 experiments. Error bars indicate standard deviations.

FIG. 15 shows a Model for the regulation of survival, proliferation and activation by the bidentate motif in the GM-CSF receptor. Shown is a schematic representation of a cytoplasmic portion of the βc subunit of the GM-CSF receptor encompassing Tyr577 and Ser585 of the bidentate motif. Low concentrations of cytokine (<3 pM) promote PKA activation, Ser585 phosphorylation, 14-3-3 binding and PI 3-kinase signalling. These signalling events are specifically linked to the regulation of cell survival only and occur in the absence of both phosphotyrosine signalling pathways and cell proliferation/activation. Increasing the concentration of cytokine results in a switch in signalling whereby Tyr577 becomes phosphorylated and a concomitant decrease in Ser585 phosphorylation occurs. This is accompanied by the binding of Shc to Tyr577, the phosphorylation of JAK2, STAT5 and ERK and the regulation of both cell survival and cell proliferation/activation. In this model, Ser585 and Tyr577 function as a molecular switch that converts an analogue input (GM-CSF concentration) to a binary output (either Ser585 signalling and survival, or Tyr577 signalling and survival together with proliferation/activation).

### DETAILED DESCRIPTION OF THE INVENTION

In a first aspect of the present invention there is provided a bidentate motif capable of binding a cytoplasmic protein and activating cellular activities in a cell, said bidentate motif comprising a tyrosine and a serine/threonine residue which are capable of interaction with cytoplasmic proteins, and wherein the residues and cytoplasmic protein can interact to activate cellular activity in the cell.

Preferably the tyrosine and serine/threonine residues function as a binary switch for independent regulation of multiple biological activities.

The present invention relates to a novel bidentate motif that is composed of two adjacent residues of tyrosine and serine which have been found to be involved in the binding of crucial cytoplasmic proteins which are involved in cell signalling pathways and which act as a binary switch intricately involved in the processes of cell survival and proliferation. In some cases, the cytoplasmic proteins are ubiquitous proteins involved in cell signalling pathways that may include mitogenesis, transformation and survival.

Many cytokines and growth factors have been shown to be potent regulators of cell survival and much effort has been devoted to mapping the intracellular signalling pathways leading to this essential biological response. However, little is known of the receptor motifs utilized by cell surface receptors to initiate the signals that promote cell survival.

The motif is bidentate by nature because of two critical amino acids, namely tyrosine and serine that are required to bind cytoplasmic proteins which will then preferably activate cascading effects of signalling systems within the cell. The tyrosine and serine are capable of phosphorylation which is important for the binding of the cytoplasmic proteins.

The term “motif” as used herein, means a distinctive amino acid sequence which is conserved and forms a unit in which the amino acids interact.

Signalling molecules may be molecules involved in cellular pathways such as but not limited to those pathways involved in proliferation, survival or differentiation. Examples of such pathways may include the JAK/STAT pathway, the ras/MAP kinase pathway or the PI-3-Kinase pathway. All pathways may be involved directly or indirectly with these functions.

The term “cell signalling pathways” as used herein includes all cellular pathways and cellular reactions which contribute to the functioning of the cell. It is not restricted to reactions that arise from cytokine mediated binding to the receptor. However, it is most preferred that the activities are activated by cytokine binding.

The cytoplasmic protein will be appropriate for the amino acid, namely tyrosine or serine, however, it is preferred that the cytoplasmic proteins that bind to the amino cells are selected from the group including 14-3-3 protein, Shc, SHIP-2, WW-domain of the prolyl isomerase, Pin1 and the ubiquitin ligase, NEDD4 and any cytoplasmic protein capable of binding a further signalling molecule which activates a cascade of events preferably leading to cell signalling pathways or other pathways and biological functions in a cell such as mitogenesis, proliferation, transformation, differentiation and cell survival. More preferably, the cytoplasmic protein is 14-3-3 or Shc. Most preferably, the 14-3-3 will bind to serine and the Shc will bind to tyrosine.

The 14-3-3 protein is a family of proteins which consists of 7 different isoforms and is expressed ubiquitously from yeast to humans. The ability of 14-3-3 to bind to a number of motifs in a wide range of signalling molecules suggests that 14-3-3 proteins may participate in a number of cell signalling pathways that may include mitogenesis, transformation and survival. Although 14-3-3 has been shown to bind a number of signalling molecules, it has been more difficult to determine how 14-3-3 can regulate signalling events.

The term “signalling molecule” is any molecule that can signal a cell signalling pathway so as to cause an activation in the signalling pathway.

Shc will bind to Tyr via its PTB domain and has the potential to both positively and negatively regulate intracellular signalling. For example, in addition to its suggested positive role in promoting signalling via the Ras/Map kinase pathway through the recruitment of grb2/sos and via the PI 3-kinase pathway through the recruitment of a grb2/GAB2/PI 3-kinase complex, Shc is also known to recruit negative regulators of signalling including the phosphatases SHP2 and SHIP.

The cytoplasmic proteins which bind to the amino acid will in turn bind to further signalling molecules which can activate a cascade of events leading to cell signalling pathways and biological functions such as, but not limited to, mitogenesis, proliferation, transformation, differentiation and cell survival or any other cytoplasmic molecule or protein which does not signal.

The bidentate motif of the present invention provides a bidentate signalling switch in a receptor with an ability to quantitatively discriminate between alternative signals. Preferably one signal regulates cell survival only and the other signal regulates cell survival and cell proliferation and functional activation. Phosphorylation of tyrosine or serine residues and the bidentate motif can be mutually exclusive enabling the receptor to generate diversity in signalling outputs but also afford signalling mechanisms that permit independent regulation of pleiotropic biological responses. The bidentate motif can be found in many cell surface receptors which shows that this phospho-tyrosine/phosphoserine bidenate may represent a generalised binary switch important in the regulation of pleiotropic responses to multiple biological stimuli.

In a preferred embodiment, the present invention provides a bidentate motif capable of binding to at least one cytoplasmic protein, said bidentate motif comprising a tyrosine and a serine/threonine residue which are capable of interaction with cytoplasmic proteins, and wherein the residue and cytoplasmic protein can interact to activate cellular activity in the cell and wherein at least one of the tyrosine or serine residues will bind to the cytoplasmic protein.

In yet another embodiment the invention provides the bidentate motif having at least one of the tyrosine or serine/threonine residues phosphorylated.

The bidentate molecule of the present invention comprises two amino acid residues which are in close proximity to each other so as to provide suitable binding and interaction of cytoplasmic proteins which may in turn bind signalling molecules and mediate cell signalling pathways. It has now been found in the present invention that the cytoplasmic proteins that bind tyrosine and serine can interact with each other. This was evident when mutations of either one of the tyrosine or serine residues of the motif only partially effected cell survival.

It is also now evidence that the tyrosine and serine can phosphorylate independently depending on the cytokine concentration. Depending on which reside is phosphorylated will affect the cytokine function on the cell. Preferably by inducing phosphorylation of the serine, cell survival is enhanced. Phosphorylation of the tyrosine enhances both cell survival and proliferation. Accordingly, the present invention provides a bidentate motif which can affect cell survival and proliferation.

In an even further preferred embodiment, the cytoplasmic proteins are Shc for tyrosine and 14-3-3 for serine such that the Shc interacts with the 14-3-3 which in turn activates a binding of a signalling molecule which then activates a cell signalling pathway. Accordingly, the present invention preferably provides a tyrosine/serine bidentate motif that is essential for cell survival and a convergence of phosphoserine and tyrosine signals through a novel Shc/14-3-3 axis.

Both amino acid residues of Tyr and Ser are available in the motif for binding. However, it is preferred that if only Tyr is bound by the cytoplasmic protein Shc, then via the Shc/14-3-3 axis, the 14-3-3 cytoplasmic protein can be activated to further activate a cell signalling pathway and subsequent biological functions. Similarly, it is further preferred that in the absence of Tyr binding to Shc, the 14-3-3 cytoplasmic protein can bind solely to the Ser585 residue and via the Shc/14-3-3 axis, the 14-3-3 protein can be activated via the Shc (unbound) cytoplasmic protein.

In a further preferred embodiment, the induction of the Shc/14-3-3 axis is via a Tyr179 on the 14-3-3 cytoplasmic protein. Applicants have found that the Tyr179 is necessary for PI-3 kinase activation as well as 14-3-3 interaction with Shc. Moreover, AKT activation also results in response to cytokine binding preferably GM-CSF stimulation.

In another aspect of the present invention, there is provided a bidentate motif capable of binding to a cytoplasmic protein comprising a tyrosine and a serine/threonine residue, said motif consisting of the following sequence alignment:
N-X-X-Y-(X)₁₋₁₃-[R/K/H/Q]-[X/Ψ]₂₋₃-S/T-X-P
wherein X is any residue, Y is tyrosine, S/T is serine or threonine and Ψ is a hydrophibic residue or an equivalent thereof.

In yet another aspect of the present invention, there is provided a bidentate motif capable of binding to a cytoplasmic protein comprising a tyrosine and a serine/threonine residue, said motif consisting of the following sequence alignment:
Y-(X)₁₋₆-[R/K/H/Q]-[X/Ψ]₂₋₃-S/T-X-P
wherein X is any residue, Y is tyrosine, S/T is serine or threonine and Ψ is a hydrophibic residue or an equivalent thereof.

The proline (p) is thought to be dispensable if the motif occurs in close proximity to the C-terminus of the protein. It is striking that in some cases this motif appears to be conserved within specific members of receptor families that regulate survival such as the FGF, low-density lipoprotein and integrin receptor families.

Preferably, the tyrosine and serine/threonine residue can react with cytoplasmic proteins and wherein the tyrosine and/or seine and their respective cytoplasmic proteins can interact to activate cellular activity in the cell. The tyrosine and serine/threonine residue can also independently phosphorylate.

More preferably, the cytoplasmic protein that binds to tyrosine and/serine are Shc, SHIP-2 and 14-3-3.

In a preferred embodiment, there is provided a binding motif of a receptor. The receptor may be any receptor that is capable of binding to an extracellular molecule or protein and which mediates its function through the binding of a cytoplasmic molecule or protein such as 14-3-3 protein or Shc, or any cytoplasmic molecule or protein capable of binding a further signalling molecule which activates a cascade of events leading to cell signalling pathways and biological functions such as mitogenesis, proliferation, transformation, differentiation and cell survival, or any other cytoplasmic molecule or protein which does not signal.

A receptor as used herein may be selected from the group including:
(1) GM-CSF/IL-3/IL-5 receptor
(2) IL6 human interleukin-6 receptor beta chain precursor (IL-6R-beta)
(3) LEPR human leptin receptor precursor (LEP-R) (OB RECEPTOR) (OB-R).
(4) TNR2 human tumor necrosis factor receptor 2 precursor (tumor necrosis factor
(5) VGR1 human vascular endothelial growth factor receptor 1 precursor
(6) TRK3 human receptor protein-tyrosine kinase TKT precursor (EC 2.7.1.112)
(7) Q01974 protein-tyrosine kinase transmembrane receptor ROR2 precursor
(8) FGR1 human basic fibroblast growth factor receptor 1 precursor (BFGF-R)
(9) Q15426 protein-tyrosine phosphatase, receptor-type, H precursor (EC 3.1.3.48)
(10) PTPM human protein-tyrosine phosphatase mu precursor (EC 3.1.3.48) (R-PTP-MU).
(11) PDGS human alpha platelet-derived growth factor receptor precursor (EC 2.7.1.112)
(12) FGR4 human fibroblast growth factor receptor 4 precursor (FGFR-4) (EC 2.7.1.112)
(13) FGR2 human fibroblast growth factor receptor 2 precursor (FGFR-2) (EC 2.7.1.112)
(14) Q13635 patched protein homolog (PTC)
(15) MANR human macrophage mannose receptor precursor.
(16) LRP2 human low-density lipoprotein receptor-related protein 2 precursor (megalin)
(17) IDD human integral membrane protein dgcr2/idd precursor (KIAA0163)
(18) AMFR human autocrine motility factor receptor precursor (AMF receptor) (gp78)
(19) ACH5 human neuronal acetylcholine receptor protein, alpha-5 chain precursor.
(20) KKIT human: stem cell growth factor receptor (proto-oncogene tyrosine-protein kinase kit) (C-KIT) (CD117 antigen)
(21) TPOR human: thrombopoietin receptor precursor (TPO-R) (myeloproliferative leukemia protein (C-MPL). TPOR or MPL.
(22) TPOR mouse: thrombopoietin receptor precursor (TPO-R) (myeloproliferative leukemia protein) (C-MPL). TPOR or MPL.
(23) Acetylcholine R
(24) Acetylcholine R alpha-5
(25) C-C chemokine receptor 6
(26) Middle T antigen
(27) integrin alpha 1
(28) FGFR2 (KGF R)
(29) FGFR1 (flg)
(30) FGFR5
(31) Erb4
(32) Vaccinia virus protein A36R
(33) Macrophage mannose R (MRC1)
(34) LDLR
(35) VLDL (rat)
(36) LRP1 low density lipoprotein receptor-related protein 1
(37) integrin beta 1
(38) integrin beta 7
(39) integrin beta 3
(40) integrin beta 5
(41) integrin beta 6
(42) G-CSFR1
(43) g-csf-r
(44) IL-6B (gp130)
(45) LeptinR
(46) ProlactinR
(47) insulinR
(48) irs-1
(49) IGFI R
(50) flt3 R
(51) VEGFR2 (FLK1)
(52) PDGF R-alpha
(53) IL-9R
(54) Beta R
(55) Neuronal acetylcholine receptor protein, alpha-3 chain
(56) protein tyrosine phosphatase receptor N
(57) glycogen synthase kinase 3 alpha
(58) p21-activated kinase 3
(59) 3-phosphoinositide dependent protein kinaes-1 (PDK1)
(60) integrin alpha 1 (laminin/collagen receptor)
   or a functional equivalent or analogue thereof.

The receptor is preferably a cytokine receptor. More preferably it is the GM-CSF/IL-3/IL-5 receptor or GM-CSF receptor.

The binding capacity of the motif may be analysed by any binding studies or experiments available to the skilled addressee. Such experiments may include measuring the binding ability of a designated cytoplasmic protein to the binding motif. For instance electrophoretic mobility shift assays (EMSA or band shift assays) or foot print assays or pull down experiments are available to measure specific binding.

Hence the bidentate motif can be identified by the presence of a tyrosine or serine residue preferably in an amino acid sequence as described above, and the ability to bind a designated cytoplasmic protein. The designated cytoplasmic protein may be 14-3-3 protein, Shc or any cytoplasmic protein capable of binding a further signalling molecule which activates a cascade of events leading to cell signalling pathways and biological functions such as mitogenesis, proliferation, transformation, differentiation and cell survival. More preferably, the cytoplasmic protein is 14-3-3 or Shc.

Preferably, the receptor is the GM-CSF/IL-3/IL-5 receptor which includes the common beta chain (β_{c}). It is found that the cytokines GM-CSF, IL-3 and IL-5 signal their actions through the surface receptor via the β_{c}. Most preferably, the binding motif comprises a sequence which includes amino acids Tyr and Ser corresponding to amino acids Tyr577 and Ser585 of the common β_{c} according to FIG. 9 or a functional equivalent or analogue thereof.

The term “functional equivalent or analogue thereof” as used herein means a sequence which functions in a similar way but may have deletions, additions or substitutions that do not substantially change the activity or function of the sequence.

The common β chain (β_{c}) is a component of a cytokine receptor and is part of a signalling subunit of the receptor. It is thought that the cytokine signals its functions through the β_{c}, initiating events which cascade and culminate in an identifiable biological function such as cell survival, proliferation, differentiation and mature cell effector functions. However, the present invention is not limited to motifs of the β_{c} but includes motifs of receptors and other proteins within the cell having similar sequences to the β_{c} and including a serine/threonine residue. It is within the scope of this invention that the bidentate motif will have the structure identified above and through this structure, the cytokine may exert its effects on the cell via the bidentate motis and the Shc/14-3-3 axis. Preferably, the bidentate motif is found in the region of the βc which includes Tyr577 and Ser585. Having this as guide all proteins having a similar motif which corresponds to the region of βc including Tyr577 and Ser585 are within the scope of this invention which defines the bidentate motif.

The region or motif comprising amino acids Tyr577 and Ser585 of the common β_{c} or functional equivalent thereof may include the residues which preferably interact with a cytoplasmic protein selected from the group including 14-3-3 protein, Shc, SHIP-2, WW-domain of the prolyl isomerase, Pin1, and the ubiquitin ligase, NEDD4 or any cytoplasmic protein capable of binding a further signalling molecule which activates a cascade of events leading to cell signalling pathways and biological functions such as mitogenesis, proliferation, transformation, differentiation and cell survival. However the present invention is not limited to this sequence but includes other equivalent sequences capable of performing the same function.

Throughout the description and claims of this specification, use of the word “comprise” and variations of the word, such as “comprising” and “comprises”, is not intended to exclude other additives, components, integers or steps.

In a preferred embodiment, the present invention provides a bidentate motif in the βc subunit of the GM-CSF, IL-3 and IL-5 receptors that is composed of two residues in close proximity to each other including Tyr577 and Ser585, which is preferably involved in the regulation of hemopoietic cell survival and proliferation. Employing a panel of retroviral constructs for the expression of wt and mutant β_{c} in primary hemopoietic cells derived from fetal livers it has now been found that this phosphotyrosine-phosphoserine bidentate motif is not only necessary for promoting cell survival and colony formation in response to GM-CSF, but that it is also sufficient for mediating these biological responses. This was evident from the inability of the βcTyr577Phe/Ser585Gly mutant to promote cell survival and colony formation in response to GM-CSF while conversely, the βcF7 mutant (in which Tyr577 and Ser585 remain intact while all 7 remaining tyrosine residues are substituted for phenylalanine) was able to promote cell survival and colony formation.

The bidentate motif may be present in any type of cell. The motif structure including the amino acid sequence as herein described can be screened in any cell. However, preferably it is a cell that can be effected by GM-CSF or includes the common βc. Most preferably, the cell is one that is effected by binding of signalling molecules to the common βc which harbours Tyr and Ser, more preferably corresponding to Tyr577 and Ser585 of the common βc. Similar bidentate motifs have been identified in a number of cell surface receptors and hence similar motifs will be involved in generating signalling switches that play important roles in their biological systems. Most preferably, the cell is a haematopoietic cell such as, but not limited to, lymphoid, myeloid and erythroid cells. Specifically, the lymphoid lineage, comprising B cells and T cells, produces antibodies, regulates cellular immunity, and detects foreign agents such as disease-causing organisms in the blood. The myeloid lineage, which includes monocytes, granulocytes, and megakaryocytes, monitors the blood for foreign bodies, protects against neoplastic cells, scavenges foreign materials, and produces platelets. The erythroid lineage includes red blood cells, which carry oxygen. Accordingly, because the bidentate motif most preferably affects the haematopoietic cell lines, it is within the scope of the present invention that cellular activities associated with any of these cell lines may also be modulated by targeting a modification to Tyr577 and/or Ser585 of the common βc of GM-CSF/IL-5/IL-3 or equivalent in another cell surface receptor.

Preferably the motif contains distinct tyrosine and serine motifs for the independent regulation of cell proliferation and cell survival. These independent biological responses was apparent in cells expressing the βcTyr577Phe/Ser585Gly mutant.

In another embodiment of the present invention, it is preferred that the motif comprises a sequence selected from any one of the following sequences:

NGPYLG...........PP..HSRSLP
NVHYRT...........P...KTHTMP
**RYFTQKEE..........TESGSGP
NKKYELQDRDVCE....P.RYRSVSEP
NPTYSVM..............RSHSYP
NIFYLIR...KSGSFPMPELKLSISFP
NEEYLDLSQ........PLEQYSPSYP
NQEYLDLSM........PLDQYSPSFP
NATYKVD...........VIQRTRSKP
NPEY................HSASSGP
NPDY.................WNHSLP
NPSYSSNPFVNYN....KTSICSKSNP
NTLY................FNSQSSP
NPVYQKTTEDEVHI...CHNQDGYSYP
NPVYLKTTEEDLSIDIG..RH.SASVG
NPTYKMYEGGEPDDVGGLLDADFALDPDKPTNFTNPVY
NPIY...............KSAVTTVV
NPLY...............KSAITTTV
NPLY...............KEATSTFT
NPLY...............RKPISTHT
NPLY...............RGSTSTFK
PGHYL...............RCDSTQP
VQTYVLQ..........GDPRAVSTQP
QVLYGQLL.............GSPTSP
HSGYRHQVPSVQVF.....SRSESTQP
WKMYEVYDA.........KS.KSVSLP
KIPYFHA..........GGS.KCSTWP
ELDYCLKGLKL......P.S.RTWSPP
SGDYMPM............SPKSVSAP
SFYYSEENKLPEPEELDLEPENMESVP(LDPSASSSSLP)
EEIYIIM.....QSCWAFDSRKRPSFP
ISQYLQN............S.KRKSRP
GTAY................GLSRSQP
***YLPQEDWAP.........TSLTRP
LVAYIAFKRWNSCKQN...KQGANSRPVNQTPPPEGEKLHSDSGIS
NVHY...........................RTPTTHTMP
NKCY..............................RGRSCP
NPNYTEFKFPQIKAHPWT.............KVFKSRTPP
NQKYMSFTSGDKSAHGYIAAHPSST.........KTASEP
NRTYYLMDPSGNAHKWCRKIQEVW........RQRYQSHP
NIFYLIRKSGSFPMPEL................KLSISFP

Preferably, these correspond to

| | |
|---|---|
| betaR .... | NGPYLG...........PP..HSRSLP |
| Acetylcholine R | NVHYRT...........P...KTHTMP |
| Acetylcholine R alpha-5 | **RYFTQKEE..........TESGSGP |
| C-C chemokine receptor 6 | NKKYELQDRDVCE....P.RYRSVSEP |
| Middle T antigen | NPTYSVM..............RSHSYP |
| integrin alpha 1 | NIFYLIR...KSGSFPMPELKLSISFP |
| FGFR2 (KGF R) | NEEYLDLSQ........PLEQYSPSYP |
| FGFR1 (fig) | NQEYLDLSM........PLDQYSPSFP |
| FGFR5 | NATYKVD...........VIQRTRSKP |
| Erb4 | NPEY................HSASSGP |
| Erb4 (second) | NPDY.................WNHSLP |
| Vaccinia virus protein A36R | NPSYSSNPFVNYN....KTSICSKSNP |
| Macrophage mannose R (MRC1) | NTLY................FNSQSSP |
| LDLR | NPVYQKTTEDEVHI...CHNQDGYSYP |
| VLDL (rat) | NPVYLKTTEEDLSIDIG..RH.SASVG |
| LRP1 low density lipoprotein | NPTYKMYEGGEPDDVGGLLDADFALDPDKPTNFTNPVY |
| receptor-related protein 1 | |
| integrin beta 1 | NPIY...............KSAVTTVV |
| interin beta 7 | NPLY...............KSAITTTV |
| integrin beta 3 | NPLY...............KEATSTFT |
| integrin beta 5 | NPLY...............RKPISTHT |
| integrin beta 6 | NPLY...............RGSTSTFK |
| G-CSFR1 (second) | PGHYL..............RCDSTQP |
| G-CSFR1 | VQTYVLQ..........GDPRAVSTQP |
| g-csf-r | QVLYGQLL.............GSPTSP |
| IL-6B (gp130) | HSGYRHQVPSVQVF.....SRSESTQP |
| leptinR. | WKMYEVYDA.........KS.KSVSLP |
| prolactinR... | KIPYFHA..........GGS.KCSTWP |
| insulinR | ELDYCLKGLKL......P.S.RTWSPP |
| irs-1 .... | SGDYMPM............SPKSVSAP |
| IGFI R | SFYYSEENKLPEPEELDLEPENMESVP (LDPSASSSSLP) |
| flt3 R | EBIYIIM.....QSCWAFDSRKRPSFP |
| VEGFR2 (FLK1) | ISQYLQN............S.KRKSRP |
| PDGF R-alpha | GTAY................GLSRSQP |
| IL-9R | ***YLPQEDWAP.........TSLTRP |
| p75 NTR | LVAYIAFKRWNSCKQN...KQGANSRPVNQTPPPEGEKLHSDSGIS |
| | (phosphorylated) |
| GM-CSF receptor βc subunit | :NGPYLGPP..........................HSRSL |
| erbB4 | :NPDY..............................WNHSL |
| fibroblast growth factor receptor 1 (fig) | :NQEYLDLSIPLD.....................QYSPSF |
| fibrobtast growth factor receptor 2 (KGF) | :NEEYLDLSQPLE.....................QYSPSY |
| fibroblast growth factor receptor 5 | :NATYKVDVI........................QRTRSK |
| low-density lipoprotein receptor-related | :NPTYKMYEGGEPDDVGGLLDADFALDPD....KPTNFTN |
| low density lipoprotein receptor | :NPVYQKTTEDEVHICHN................QDGYSY |
| very low density lipoprotein receptor | :NPVYLKTTEEDLSIDIG................RHSASV |
| Neuronal acetylcholine receptor protein, | :NVHY...........................RTPTTHTM |
| protein tyrosine phosphatase receptor N | :NKCY..............................RGRSC |
| glycogen synthase kinase 3 alpha | :NPNYTEFKFPQIKAHPWT.............KVFKSRTP |
| p21-activated kinase 3 | :NQKYMSFTSGDKSAHGYIAAHPSST.........KTASE |
| 3-phosphoinositide dependent protein | :NRTYYLMDPSGNAHKWCRKIQEVW........RQRYQSH |
| integrin alpha 1 (laminin/collagen) | :NIFYLIRKSGSFPMPEL................KLSISF |
| integrin beta 1 (integrin VLA-4 beta) | :NPIY............................KSAVTTV |
| integrin beta 3 (platelet glycoprotein IIIa) | :NPLYKEA..........................TSTFTN |
| integrin beta-6 | :NPLYR............................GSTSTF |
| integrin beta-7 | :NPLYKS...........................AITTTI |
| MOTIF (forward) | n-X-X-Y-X (3, 17) - [RKHQ] -X (2, 3) - [ST] -X-P |
| EGFR | RYSSDPTGALTEDSIDDTFLPVPEYINQSVPKRPAGSVQNPVY.. (NPEY) |
| Erb2 | KTLSPGKNGVVKDVFTF.................GGAVENPEY |
| Voltage-depend | RTHSLP.................................NDSY |
| T-type Ca chan. | |
| alpha-1G subunit | |
| EPO R | SDGPYSNPYENSLIPAAEPLPPSYVACS |
| | (Y NB in P1 3-K; S is end of protein, JBC 270: 23402) |
| MOTIF (reverse) | [EKHQ] -X (2, 3) - [ST] -X-P-X (0, 33) -N-X-X-Y |
| TRHR receptor | HFSTELD |
| IL-2R beta | NQGYFFFHLPDALEIEACQVYFTYDPYSEEDPDEGVAGAPTGSSP |

The present invention has found that mutation of both Ser585 and Tyr577 is required to abolish hemopoietic cell survival in response to GM-CSF would suggest that these residues are able to independently regulate cell survival and that Tyr577 can compensate for the loss of Ser585 in the βcSer585Gly mutant while Ser585 can compensate for the loss of Tyr577 in the βcTyr577Phe mutant. Without being limited by theory, it is postulated that 14-3-3 is able to bind Ser585 and Shc is able to bind Tyr577 simultaneously on the same βc and that each adaptor or scaffold protein is able to regulate an intracellular signalling pathway that promotes cell survival. This bidentate configuration interacts to activate cellular activities.

The 14-3-3 pathway regulated by Ser585 and the Shc pathway regulated by Tyr577 have the potential to signal through PI 3-kinase. Thus, the ability of Ser585 and Tyr577 to compensate for each other may be due to their respective ability to function as a bidentate scaffold that is able to regulate PI3-kinase through two alternate pathways.

The present invention therefore also provides an interaction between Shc and 14-3-3 such that the amino acid residues Tyr and Ser can exert their effects independently via the interaction between Shc and 14-3-3. Either Tyr or Ser activation (or phosphorylation) can activate Shc or 14-3-3 and cause the flow on effects to the signalling pathways via 14-3-3.

It is further found that Shc interacts with 14-3-3 via a Tyr 179 with is necessary for PI-3 kinase activity. Via this signalling molecule, further signalling pathways are activated leading to cellular activities such as mitogenesis, proliferation, transformation, differentiation, cell survival, chemotaxis, motility, enhanced phagocytosis, bacterial killing, superoxide production and cytotoxicity.

In another aspect of the present invention there is provided a method of modulating cellular activities in a cell, said method comprising
modifying phosphorylation of a Tyr and/or Ser residue of a bidentate motif capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

The modulation of the phosphorylation events which phosphorylate the tyrosine and/or serine residue on the bidentate motif will affect the binding of a cytoplasmic protein which in turn will affect the activation of signalling molecules which activate a cascade of events leading to cell signalling pathways and cellular activities. Preferably the cellular activities are selected from the group including mitogenesis, proliferation, transformation, differentiation, cell survival, chemotaxis, motility, enhanced phagocytosis, bacterial killing, superoxide production and cytotoxicity. Most preferably, the cellular activity is proliferation or cell survival. More preferably it is cell survival.

Therefore the modulation of the cellular activity is dependent upon the ability of the residues to bind to cytoplasmic proteins. The phosphorylating events are critical for binding the cytoplasmic proteins to the tyrosine and/or serine. Both need not be phosphorylated nor bind to their respective cytoplasmic proteins. The effect of the binding may be further communicated via the Shc/14-3-3 axis. Either one of the cytoplasmic proteins needs to be activated to exert the effect via 14-3-3 which in turn can activate the PI-3 kinase through the Tyr179.

Hence the initiation of the regulating effects depend on the phosphorylation of the Tyr and/or Ser of the bidentate motif as described above.

“Modulation” or “Modulating” as used herein with respect to cellular activities means modifying or altering the activity compared to unmodified levels. The activity may be increased or decreased. For instance, proliferation may be increased or decreased. The modulation may cause an enhancement or reduction of the cellular activity.

The modification of phosphorylation of the Tyr or Ser may be an increase or a decrease of the phosphorylation of the residue. Methods of increasing or decreasing (inhibiting) phosphorylation may be known to those skilled in the art. However, specifically, the use of specific kinase inhibitors are preferred to inhibit the phosphorylation.

In one preferred embodiment, the modification of phosphorylation is by inducing a mutation at the position of Tyrosine or Serine of the common βc. More preferably the mutation is at a position equivalent to Tyr577 or Ser585 of the common βc. The mutation may include a substitution, deletion, or insertion of another amino acid such that the position of Tyr or Ser is debilitated.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

“Deletions” result from the amino acid being physically removed. The position may be targeted by methods available to the skilled addressee as used in site directed mutagenesis.

“Insertions” may arise where a similar amino acid is not inserted but another amino acid is inserted. Hence it is a non-conservative amino acid change.

Preferably, the substitutions replace Tyr or Ser, preferably Tyr577 or Ser585 of the common βc with another amino acid. Preferably the Tyr is replaced with phenylalanine, more preferably Tyr577Phe and the Ser is replaced by Gly, more preferably, Ser585Gly.

In another preferred embodiment the modification of phosphorylation is by the introduction of a cytokine to the binding motif. Preferably the phosphorylation occurs at a position equivalent to Tyr577 or Ser585 of the common beta chain (βc). Preferably, the cytokine introduced is GM_CSF. Depending on the concentration of the cytokine, phosphorylation may increase or decrease.

Applicants have found that <3 pM GM-CSF was able to promote Ser585 phosphorylation, 14-3-3 binding and the recruitment of the p85 subunit of PI 3-kinase while >10 pM GM-CSF resulted in a switch in signalling. This switch was manifested by the appearance of Tyr577 phosphorylation, a concomitant decrease in Ser585 phosphorylation and the phosphorylation of JAK2, STAT5 and ERK (FIG. 11). Hence to phosphorylate serine, a concentration of GM-CSF of up to 10 pM is preferred. More preferably the concentration is 3 pM. To phosphorylate tyrosine, it is preferred to use GM-CSF greater than 10 pM. Hence it is most preferred to use this cytokine at least 10 pM.

In another aspect of the present invention, there is provided a method of activating cellular activities in a cell said method including:
inducing phosphorylation of a bidentate motif, a functional equivalent or analogue thereof capable of binding a cytoplasmic protein and activating cellular activities in a cell, said bidentate motif comprising a tyrosine and a serine/threonine which are capable of interaction with cytoplasmic proteins, and wherein the residue and cytoplasmic protein can interact to activate cellular activity in the cell and
subjecting the bidentate motif to a cytoplasmic protein to bind to the Tyr and/or Ser.

Preferably the cytoplasmic protein is 14-3-3 protein or Shc, or any cytoplasmic protein capable of binding a further signalling molecule which activates a cascade of events leading to cell signalling pathways and biological functions such as mitogenesis, proliferation, transformation, differentiation cell survival, chemotaxis, motility, enhanced phagocytosis, bacterial killing, superoxide production and cytotoxicity. Preferably the cytoplasmic binding protein is 14-3-3 or Shc.

The 14-3-3 or the Shc molecule binds not only to the bidentate motif via serine or tyrosine respectively, but has the ability to bind to a wide range of signalling molecules and to participate in a number of cell signalling pathways resulting in mitogenesis, transformation, differentiation cell survival, chemotaxis, motility, enhanced phagocytosis, bacterial killing, superoxide production and cytotoxicity.

Once 14-3-3, Shc or an equivalent binds to the bidentate motif, their ubiquitous nature can bind cytoplasmic proteins involved in signalling pathways which activate these pathways. The binding motif is phosphorylated and preferably the ⁵⁸⁵Ser and/or ⁵⁷⁷Tyr or equivalent residue is phosphorylated. 14-3-3 and/or Shc can bind to the phosphorylated motif via these residues thereby positioning the 14-3-3 and the Shc close for further binding of cytoplasmic proteins involved in cell signalling (signalling molecules) for cellular activities such as proliferation, differentiation, cell survival, chemotaxis, motility, enhanced phagocytosis, bacterial killing, superoxide production and cytotoxicity.

The cell as used herein may be an isolated cell or be a cell contained within tissue or bodily fluids. Preferably the cell is a haematopoetic cell as herein described. Generally the cell will have a receptor for a cytokine such as the GM-CSF. Most preferably the cell will have the GM-CSF receptor and activate via the βc.

Inducing phosphorylation may include any means to phosphorylate the cell or the tyrosine or serine as described herein. The cell may be directly subjected to phosphorylating agents. A kinase is preferred or the cell may be subjected to cytokines, preferably GM-CSF at a concentration suitable to induce phosphorylation of the serine or tyrosine residue.

In yet another aspect of the present invention there is provided a method of modulating cellular activities in a cell, said method including:
modifying phosphorylation of a bidentate motif, a functional equivalent or analogue thereof capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell;
subjecting the bidentate motif to a cytoplasmic protein which binds to the tyrosine and serine residue; and
activating a cell signalling pathway by interacting the bound cytoplasmic protein with a signalling molecule involved in the pathway.

Preferably, the cytoplasmic protein is 14-3-3 protein, Shc or any cytoplasmic protein capable of binding a further signalling molecule which activates a cascade of events leading to cell signalling pathways and biological functions such as mitogenesis, proliferation, transformation, differentiation, cell survival, chemotaxis, motility, enhanced phagocytosis, bacterial killing, superoxide production and cytotoxicity. Preferably the cytoplasmic binding protein is 14-3-3 or Shc.

There are many signalling molecules involved in cellular pathways leading to cellular activity. However, it is preferred in the present invention to provide a molecule that binds to a phospho-serine bound 14-3-3 molecule or a phosphotyrosine bound Shc such that a pathway is coupled to the motif or equivalent unit in a receptor and brought into close proximity to downstream signalling proteins at, or near, the cell membrane. Cellular activities may include cell survival, proliferation, transformation, differentiation, mitogenesis, chemotaxis, motility, enhanced phagocytosis, bacterial killing, superoxide production and cytotoxicity.

Preferably the cellular activity is cell survival. Either one or both of the Tyr or Ser residues may be phosphorylated in the bidentate motif. Preferably, serine is phosphorylated at low concentrations of GM-CSF to induce cell survival. Preferably the concentration of GM-CSF is up to 10 pM. Preferably the concentration is 3 pM. More preferably the concentration is 1 pM. Alternatively, tyrosine may be phosphorylated preferably using GM-CSF preferably at a concentration of at least 10 pM.

Phosphorylation of the motif may be modified by any means which results in inhibition or activation of the phosphorylation of the bidentate motif. Preferably, the Ser⁵⁸⁵ or the Tyr⁵⁷⁷ residue of the βc of a GM-CSF/IL-5/IL-3 is modified by phosphorylation.

For regulating cell survival, it is preferred to activate the PI-3-kinase pathway using a PI-3 kinase bound to a phosphoserine bound 14-3-3.

Regulation of cell survival may include enhancing or reducing cell survival or even causing cell death. This may be achieved by enhancing or inhibiting any of the steps herein described. For instance enhancing phosphorylation of the bidentate motif may enhance survival. Alternatively, inhibiting phosphorylation may inhibit cell survival. Phosphorylation of one or the other of the Tyr or Ser is necessary for cell survival. Abolishing phosphorylation of both abolishes cell survival.

Preferably, the cellular activity is cellular proliferation. Applicants have found that by increasing phosphorylation of tyrosine, preferably with GM-CSF, can increase proliferation and cell survival. Increased GM-CSF concentration of at least 10 pM can increase tyrosine phosphorylation with decreased serine phosphorylation.

Using such a binary switch for the independent regulation of cell survival and proliferation/activation is likely to have a number of biological advantages. Firstly, the generation of two distinct and mutually exclusive signals would allow the cellular machinery that regulate cell survival and proliferation/activation to unambiguously interpret incoming signals without the need to evaluate the signal strength or the time of signal duration. Secondly, apart from some notable exceptions, many cells in adult metazoa spend the greater proportion of their lives in a quiescent, non-activated state and would therefore require signalling strategies that allow the regulation of long-term survival in the absence of proliferation or activation. For example, the long-term survival of myeloid cells such as monocytes/macrophages and dendritic cells in peripheral tissues is critical in their function as sentinels that guard against foreign pathogens. The results presented in this application would suggest that the long-term survival of these cells in peripheral tissues in the absence of proliferation or activation would require Ser585 phosphorylation. In the event of invading foreign pathogens an emergency response would be triggered and increased cytokine concentrations would result in a switch in signalling with a decrease in Ser585 phosphorylation, an increase in Tyr577 phosphorylation and the regulation of both survival and activation of a proliferation/differentiation program in hemopoietic progenitor cells in the bone marrow and of survival and activation of mature neutrophils and monocytes in the periphery. Employing a bidentate motif may provide a failsafe and noise-free mechanism that not only allows the independent regulation of long-term survival but may also reduce the likelihood of unscheduled cell proliferation or activation that are typical in cancer and inflammation.

Thirdly, there is evidence that at the level of an individual cell, many biological responses are in fact binary.

In another preferred aspect of the present invention, there is provided a method of inhibiting cell survival, said method including inhibiting the binding of a cytoplasmic protein to a bidentate motif capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

Preferably the cytoplasmic protein is 14-3-3 and/or Shc which bind to Ser or Tyr respectively. The binding may be inhibited in one or both.

Preferably the bidentate motif is part of a receptor.

Preferably the receptor is the GM-CSF/IL-5/IL-3 receptor although a phosphorylation event which phosphorylates ⁵⁸⁵Ser or ⁵⁷⁷Tyr of the common μc may also trigger the binding of 14-3-3 or Shc to the motif.

Inhibiting the binding of the cytoplasmic protein to the receptor may be achieved by inhibiting phosphorylation of the Tyr and/or Ser, mutating the Tyr and/or Ser or using antagonists.

Antagonists that bind to the bidentate motif wherein the motif is in either the phosphorylated or unphosphorylated form may be useful to inhibit cell survival or activation. Preferably antagonists may be useful to inhibit cell survival or activation by preventing phosphorylation preferably by preventing serine and/or tyrosine, preferably an equivalent Tyr577 or Ser585 of the common βc, phosphorylation of the β_{c} or equivalent thereby preventing the cytoplasmic protein binding to the bidentate motif. Alternatively, they may prevent the interaction of a signalling molecule binding to a phosphoserine bound 14-3-3 or phosphotyrosine Shc equivalent. Prevention of phosphorylation of the β_{c} or bidentate motif as herein described may be by inhibition of the specific kinases involved in the phosphorylation of the serine/threonine or tyrosine residue or it may include mutation of the bidentate motif to prevent the cytoplasmic protein such as 14-3-3 or Shc from binding and activating cell cycle pathways. Kinase inhibitors such as H89 which binds to PKA may be used.

Antagonists may include antibodies, small peptides, small molecules, peptide mimetics or any type of molecule known to those skilled in the art that are directed to the bidentate motif so as to prevent attachment of cytoplasmic proteins such as 14-3-3 to a phosphoserine residue or a Shc to a phosphotyrosine residue or a signalling molecule. Antibodies may be generated in response to any of the bidentate motifs described above by methods known and available to the skilled addressee.

Hence, the antagonists as described may be useful as cancer therapeutics to prevent cell survival of cancer cells or cell activation such as myeloid cell activation and may be useful for preventing or treating leukaemia such as acute myeloid leukaemia (AML). Other uses of antagonists may be in prevention and treatment of inflammatory diseases.

This may be useful to prevent those functions related to cell activation, particularly myeloid cell activation. The functions may be selected from the group including chemotaxis, motility, enhanced phagocytosis, bacterial killing, superoxide production and cytoxicity. These functions may also contribute to inflammation including, but not limited to, asthma and rheumatoid arthritis.

In another aspect of the invention, there is provided a method of inhibiting cell activation, said method including inhibiting binding of a cytoplasmic protein to a bidentate motif, a functional equivalent or analogue thereof capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

This method of interaction may be a useful tool for a method of treating or preventing cell proliferative diseases such as AML or cancer.

Inhibition may be by way of the use of antagonists as herein described, or inhibition of phosphorylation of the bidentate motif or by any means that prevents activation of cell cycles via the bidentate motif described in the present invention. Methods of inhibiting phosphorylation using cytokines such as GM-CSF are herein described. High concentrations of GM-CSF can affect phosophorylation of the serine. However, at high concentrations of GM-CSF, it may not affect cell survival since the tyrosine may phosphorylate to enhance phosphorylation and cell survival. Cell survival induced at low GM-CSF concentration may be affected by the concentration of the cytokine.

It is also preferred to target the interaction between the cytoplasmic protein Shc and 14-3-3. It has been found that Shc interacts with 14-3-3 via a Tyr179 on the 14-3-3 molecule. Targeting or inhibiting this interaction may prevent the action of 14-3-3 to further activate and interact with triggering molecules such as PI-3 kinase which activates the PI-3 kinase pathway.

Also, it is preferred that where one or the other of Tyr or Ser is phosphorylated in the absence of the other, the cell will rely in the interaction between Shc and 14-3-3. Therefore by interfering with this interaction, further cell activations are interfered with.

In another aspect, there is also provided a method of treating a cytokine mediated condition in a cell said method comprising:
regulating activation of phosphorylation of a bidentate motif, a functional equivalent or analogue thereof capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

The cytokine mediated condition is a condition which requires a cytokine to bind to its receptor to induce a cellular activity. By regulating the activation, cellular activity may be activated to increase the phosphorylation or to decrease phosphorylation depending on the condition to be treated. Preferably, the cytokine mediated condition is a GM-CSF/IL-5/IL-3 mediated condition and the bidentate motif includes the amino acids ⁵⁷⁷Tyr and ⁵⁸⁵Ser of the common βc.

In the present invention it is shown that Tyr577 and Ser585 function to promote hemopoietic cell survival in response to GM-CSF. Ser585 and Tyr577 may also function independently of each other in a non-redundant manner. In terms of a specific role for Ser585, GM-CSF was able to promote colony formation, cell survival and cell proliferation in cells expressing the βcSer585Gly mutant in the presence of 10% FCS, a defect was found in cell survival when experiments were performed in low serum concentrations (i.e. 0.1% FCS). These results would suggest that under reduced serum conditions where the concentration of survival cytokines and growth factors is low, Ser585 plays an essential role in regulating hemopoietic cell survival in response to GM-CSF. Thus, it is possible that the Ser585 pathway is important in regulating GM-CSF-mediated hemopoietic cell survival in vivo under conditions where the concentrations of other cytokines and growth factors is limiting. Such conditions would presumably not include myeloid cells at sites of inflammation where the extracellular milieu is known to contain, in addition to GM-CSF, high concentrations of other cytokines and growth factors. However, myeloid cells such as monocytes/macrophages and dendritic cells are known to reside in peripheral tissues not undergoing inflammation where they are thought to function as sentinels on guard against foreign pathogens. The long-term survival of these cells in the periphery where the concentrations of cytokines is likely to be more limiting may be dependent on the ability of GM-CSF to promote signalling via the Ser585 pathway.

In terms of a specific role for Tyr577, cells expressing the βcTyr577Phe mutant demonstrated increased colony formation and colony size in response to GM-CSF. In addition, this receptor mutant promotes increased hemopoietic cell survival. These findings using primary hemopoietic cells are the first to demonstrate a biological role for Tyr577 and would suggest that this residue plays a negative role in βc signalling. The adaptor protein Shc, which binds Tyr577 via its PTB domain, has the potential to both positively and negatively regulate intracellular signalling. For example, in addition to its suggested positive role in promoting signalling via the Ras/Map kinase pathway through the recruitment of grb2/sos and via the PI 3-kinase pathway through the recruitment of a grb2/GAB2/PI 3-kinase complex, Shc is also known to recruit negative regulators of signalling including the phosphatases SHP2 and SHIP. Alternatively, Tyr577 may bind other PTB domain proteins that negatively regulate receptor function, however, the direct binding of other PTB domain proteins has not been reported.

These contrasting roles for receptor tyrosine phosphorylation and Ser585 phosphorylation may have important consequences in carcinogenesis. Considerable effort is presently being devoted to developing tyrosine kinase inhibitors for the treatment of cancer. However, the results presented herein would suggest that these inhibitors may prove highly effective in blocking the proliferation of transformed cells but not in blocking survival allowing a reservoir of cancer cells to persist. Such an example can be seen in CML patients following treatment with the tyrosine kinase inhibitor, imatinib, which blocks BCR-ABL. While imatinib has demonstrated an impressive ability to achieve a hematological response in CML, approximately 82% of patients with myeloid blast crisis relapse within 3 months. Lack of eradication, due to the long-term survival of transformed cells may permit secondary mutations and the emergence of drug-resistant clones. The constitutive Ser585 phosphorylation observed in myeloid leukemias examined in our studies (FIG. 12) raises the possibility that at least part of the transformed phenotype of some leukemias could be due to a deregulated survival switch mechanism causing constitutive cell survival.

Cell surface receptors with seemingly disparate biological functions are commonly observed to utilize similar strategies for the transduction of intracellular signals. For example, receptor tyrosine phosphorylation and the recruitment of SH2 and PTB domain proteins is a widely employed mechanism to physically couple activated receptors to downstream signalling pathways. Therefore, the finding that similar tyrosine/serine bidentate motifs occur in a wide variety of cell surface receptors would suggest that the βc may be a prototypic example of a novel signalling device employed for the regulation of specific biological responses to other cytokines and growth factors.

This binary switch is deregulated in at least some leukemias. Hardwiring such a molecular switch into cell surface receptors may permit the signals that promote cell survival to be uncoupled and independently regulated from those that regulate proliferation/functional activation. Similar bidentate motifs have been identified in other cell surface receptors suggesting that such signalling switches may play important roles in generating specificity and pleiotropy in other biological systems.

In another aspect, there is provided a method for diagnosing a proliferative condition involving cell proliferation or cell survival, said model including:
detecting a level of phosphorylation of Tyr and/or Ser in a bidentate motif in a cell; and
comparing against a cell of a normal level of phosphorylation.

The present invention may also be used in a method for diagnosing proliferative conditions involving cell proliferation and cell survival. Given the interaction between the bidentate motif and the cytoplasmic proteins, any part of the interactions can be monitored to determine any aberration between the cells in question and that of a normal cell. A normal cell generally obtained from a control subject who does not display a proliferative condition will have a “normal level of phosphorylation”. Aspects of the method may analyse the phosphorylation ability of the Tyr or the Ser residues or analyse the interaction between the respective cytoplasmic proteins of Shc and 14-3-3 both with Tyr or Ser or between themselves.

As described herein, phosphorylation of the serine or tyrosine may have differing affects on the outcome of survival or proliferation. Detection of the phosphorylation state can determine and diagnose the status of the cell.

A model may also be based on the activity of the 14-3-3 molecule and the activity or phosphorylation ability of the Tyr179 of the 14-3-3. Aberrations in any of these points may indicate a disorder in proliferation ability of the cell.

Preferably the method is based on a haematopoetic cell as described above. More preferably, the cell has a GM-CSF receptor including the Tyr577 and Ser585 of the common βc upon which the model is based.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

Examples of the procedures used in the present invention will now be more fully described. It should be understood, however, that the following description is illustrative only and should not be taken in any way as a restriction on the generality of the invention described above.

### EXAMPLES

The following procedures have been used in the Examples described below:

(a) Transduction of Primary Hemopoietic Cells

The ability of wild type (wt) and mutant β_{c} to transduce specific functional responses was assessed in primary mouse hemopoietic cells derived from fetal livers which exhibit the full plethora of biological responses to GM-CSF. Fetal liver cells were harvested from E13.5 mice and transduced with bicistronic retroviral constructs for the expression of both the GMRα and the βc subunit of the human GM-CSF receptor. To avoid crosstalk between endogenous mouse β subunits and transduced human βc subunits (McClure et al., 2001), we have transduced human GM-CSF receptors into a β subunit null background using fetal liver cells derived from βc −/− βIL-3 −/− double knockout SV129 mice (Robb et al., 1995). Transductions were performed as previously described (Le et al., 2000) using Ψ2 packaging cell lines stably transfected with pRUF-IRES-GMRαβc (wt GM-CSF receptor), pRUF-IRES-GMRαβcTyr577Phe, pRUF-IRES-GMRαβcSer585Gly, pRUF-IRES-GMRαβcSer585Gly/Tyr577Phe, pRUF-IRES-GMRαβcF8 (all cytoplasmic tyrosines substituted for phenylalanine), or pRUF-IRES-GMRαβc-addback (a βc mutant in which Tyr577 and Ser585 remain intact while all remaining tyrosines were substituted).

(b) Cell Survival Assays

Survival was determined by annexin V-FITC (Roche) staining essentially as previously described (Guthridge et al., 2000). Briefly, transduced fetal liver cells were plated in IMDM and either 0.1% or 10% FCS containing either no GM-CSF, 3.3 μM hGM-CSF or a positive control cytokine cocktail containing 50 ng/ml IL-6 (Peprotech), 100 ng/ml mouse SCF and 10 ng/ml G-CSF (Amgen). After 48 hours, cells were stained with the 4H1 anti-GMRα mAb and annexin V-FITC. The proportion of GMRα-positive cells remaining viable (annexin V-FITC-negative) after culture was analysed by flow cytometry. The regulation of TF-1 cell survival by GM-CSF was determined as previously described (Guthridge et al., 2000; Guthridge et al., 2004). To examine the survival of primary human neutrophils in response to GM-CSF, we modified our previously described assay (Begley et al., 1986). Neutrophils purified by centrifugation through Lymphoprep (Axis-Shield) were plated at 1.5×10⁵ cells/ml in IMDM/0.5% FCS containing different concentrations of GM-CSF and cell survival was examined as previously described (Guthridge et al., 2000). In some experiments forskolin ICN Biochemicals), H89 (Seikaguku Corporation), LY294002 (Cayman Chemical Company), genestein (Sigma) or AG490 (Biomol) were used.

(c) CD11b Expression in Human Neutrophils

Purified neutrophils were stimulated with GM-CSF in 1 ml Hepes buffer at 2.5×10⁴ cells/sample for 75 minutes at 37° C., then stained with an anti-human CD11 b-RPE mAb (DakoCytomation) as recommended by the manufacturer and analysed for CD11b surface expression by flow cytometry.

(d) Cell Proliferation Assays

Cell cycle progression was determined by BrdU incorporation as previously described (Guthridge et al., 2004) using the in situ cell proliferation kit (Roche). Briefly, fetal liver cells transduced with either the wt or mutant GM-CSF receptors were firstly stained with the 4H1 anti-GMRα mAb and purified using fluoresence activated cell sorting before plating in IMDM and 10% FCS containing either no GM-CSF, 3.3 μM hGM-CSF or a positive control cytokine cocktail composed of IL-6, SCF and G-CSF (see above) for 24 hr with the cells being pulsed with 10 μM BrdU (Roche) for the last 4 hours. Cells were then fixed and stained with anti-BrdU-FITC antibodies and BrdU incorporation assessed by flow cytometry. TF-1 cell proliferation was also assessed by BrdU incorporation essentially as previously described (Guthridge et al., 2004).

(e) Colony Assays

Fetal liver cells were transduced with wt and mutant human GM-CSF receptors and plated at 10⁵ cells/dish in 0.3% agar and colony forming cells were assayed as previously described (Peters et al., 1996). Groups of cells containing greater than 40 cells were counted as colonies.

(f) Peptide Pull-Downs

Pulldown experiments were performed as previously described (Stomski et al., 1999). Peptides were synthesized with a biotin-N-Hydroxysuccinimide (biotin-NHS)N-terminus and were HPLC purified (Mimotopes, Victoria). Peptide sequences were biotin-NHS-KGGFDFNGPYLGPPHSRSLPDGG (non-phospho-Tyr577/non-phospho-Ser585), biotin-NHS-KGGFDFNGP(pY)LGPPHSRSLPDGG (phospho-Tyr577/non-phospho-Ser585), biotin-NHS-KGGFDFNGPYLGPPHSR(pS)LPDGG (non-phospho-Tyr577/phospho-Ser585) and biotin-NHS-KGGFDFNGP(pY)LGPPHSR(pS)LPDGG (phospho-Tyr577/phospho-Ser585).

(g) Immunoblotting

Cells were factor-deprived in RPMI containing 0.1% FCS for 24 h and then stimulated for 10 min with different GM-CSF concentrations before lysis as previously described (Guthridge et al., 2000). Human mononuclear cells from normal donors and from leukemic patients were separated from peripheral blood by centrifugation over ficoll. The βc subunit was immunoprecipitated using a mixture of 1C1 and 8E4 anti-βc mAb (1C1/8E4)(1 μg each/immunoprecipitation) and immunoprecipitates subjected to SDS-PAGE and immunoblot analysis. Affinity purified anti-14-3-3 pAb (EB1) were used at a dilution of 1:5000 (Guthridge et al., 2000). Anti-MAP2 mAb (MK12)(Pharmingen) was used at a dilution of 1:5000. Anti-p85 pAb antibodies (UBI) were used at a dilution of 1:1000. The 4G10 anti-phosphotyrosine mAb (UBI) was used at 1 pg/ml. The anti-active-ERK pAb (Promega) was used at 50 ng/ml, the anti-phosphorylated STAT5 mAb (Zymed) was used at 2 μg/ml, the anti-phospho-JAK2 pAb (Affinity Bioreagents) was used at 0.5 μg/ml. Affinity purified anti-β_{c} phospho-577 pAb (Guthridge et al., 2004) were used at a dilution of 1:1000. Antibodies to phospho-Ser585 of β_{c} (Guthridge et al., 2000) were used at a dilution of 1:5000. The phosphorylation of Akt in response to GM-CSF was examined in CTL-EN cells electroporated with the pCMV-AKT-HA plasmid (30 μg DNA/1×10⁷ cells)(kindly provided by P. N. Tsichlis). 24 h after electroporation the cells were factor-deprived overnight in DMEM/0.5% FCS. Cells were then stimulated (10⁷ cells/time point) with either 1 pM or 1000 pM GM-CSF and lysed in RIPA buffer (150 mM NaCl, 1.0% NP40, 0.5% Deoxycholate, 0.1% SDS, 50 mM Tris pH8.0) and subjected to SDS-PAGE and immunoblot analysis with the anti-phospho-Akt Ser473 pAb (1:1000)(Cell Signalling Technologies).

(h) Protein Kinase A (PKA) Kinase Assay

TF-1 cells were factor-deprived overnight in DMEM containing 0.5% FCS. Cells (4×10⁶) were stimulated with GM-CSF or forskolin before lysis in 1 ml 20 mM Tris HCl pH 7.4, 0.5 mM EDTA, 0.5 mM EGTA, 10 mM 2-mercaptoethanol, 5% Glycerol and 2 mM NaF by 20 strokes of a dounce homogenizer on ice. The lysate was centrifuged at 170 g for 15 min and the supernatant then centrifuged at 100,000 g for 1 h at 4° C. The resulting pellet was resuspended in NP40 lysis buffer with a protease/phosphatase inhibitor cocktail (Guthridge et al., 2000; Guthridge et al., 2004). Lysate (2 μl) was then tested for PKA activity by adding 40 μM kemptide, 0.25 μCi [y-³²P] ATP, 1 μM cold ATP, in a buffer containing 10 mM MgCl₂, 10 mM Tris HCl pH7.4, 1 mM β-glycerol phosphate and 1 mM DTT. Reactions (20 μl) were incubated at 30° C. for 30 min and aliquots were examined for 32P-labelled kemptide on p81 phosphocellulose filters (Whatman) and liquid scintillation counting (Guthridge et al., 2000).

(i) Bioinformatic Analysis

We performed Blast searches (National Center for Biotechnology Information; www.ncbi.nlm.nih.gov) and Scansite searches (http://scansite.mit.edu) to identify signalling proteins that contained a PTB binding site (N-X-X-Y)(Laminet et al., 1996) adjacent to a 14-3-3 binding site ([R/K/H/Q]-[X]₂₋₄-S-X-P (Yaffe and Elia, 2001). Those motifs in proteins that were conserved between the human, mouse and rat homologues were retrieved.

### Example 1

### Role of Ser585 on βctyrosine Residues in the Ability of GM-CSF to Regulate Primary Haematopoietic Cell Function

a) Transduction of Primary Fetal Liver Cells

The ability of wt and mutant βc to transduce specific biological responses following GM-CSF stimulation was assessed in primary mouse hemopoietic cells derived from fetal livers. Fetal liver cells were harvested from E12.5 mice and transduced with bicistronic retroviral constructs for the expression of both the α subunit (GMRα) and the βc subunit of the human GM-CSF receptor. Human GM-CSF receptors were transducted into a β subunit null background using fetal liver cells derived from βc −/− β_{IL-3} −/− double knockout SV129 mice. For these transductions, Ψ2 retroviral packaging cell lines were firstly stably transfected with constructs for the wild type GM-CSF receptor (pRUF-IRES-GMRαβc) and mutant receptors (pRUF-IRES-GMRαβcTyr577Phe, pRUF-IRES-GMRαβcSer585Gly, pRUF-IRES-GMRαβcSer585Gly/Tyr577Phe, pRUF-IRES-GMRαβcF8 and pRUF-IRES-GMRαβcF7. Pools of stably transfected viral-producing cell lines were then used to transduce primary hemopoietic cells derived from fetal livers. Transductions were set up by co-culturing irradiated (30Gy) Ψ2 cells (8×10⁵/ml) with fetal liver cells (**/ml) in IMDM, 15% heat inactivated fetal calf serum (HI FCS), 0.5 ug/ml polybrene, 0.2 ng/ml mouse SCF. After 48 hours loosely adherent fetal liver cells were harvested by gently shaking and the cells transferred to a fresh flask overnight prior to use in functional assays.

b) Colony Assays

The ability of GM-CSF to promote colony formation is dependent of the ability of the βc to promote two critical biological responses; cell survival and proliferation. The lack of colonies observed for the βcTyr577Phe/Ser585Gly and the βcF8 mutant could be due to a defect in their regulation of either cell survival, cell proliferation or both survival and proliferation. To examine these individual possibilities, we examined the ability of the wt and mutant βc to specifically promote either cell survival or proliferation in response to GM-CSF. For the survival assays, fetal liver cells as prepared in Example 1 were transduced with wt and mutant βc and plated out in either no factor, 50 ng/ml GM-CSF or control cocktail as a positive control for cell viability. After 24 or 48 hours, cells were double-stained firstly for GM-CSF receptor expression using the 4H1 anti-GMRα monoclonal antibody and an anti-mouse-PE antibody, and secondly for apoptosis using annexin-V-FITC. Cells expressing the GM-CSF receptor (4H1 positive) were examined for cell viability (annexin-V-FITC negative) by flow cytometry and the results are shown in FIG. 3. While GM-CSF was able to promote the survival of fetal liver cells transduced with the wtβc, βcTyr577Phe, βcSer585Gly, βcF8 and the βcF7 mutants, survival of cells expressing the βcTyr577Phe/Ser585Gly was dramatically reduced. The combined observations that the βcTyr577Phe/Ser585Gly mutant was defective in promoting survival whilst the βcF7 mutant (where Tyr577 and Ser585 are intact) was able to promote cell survival would suggest that these residues constitute a distinct motif that is both necessary and sufficient for regulating hemopoietic cell survival. Furthermore, these results would indicate that the lack of colonies observed in FIG. 2 for cells expressing the βcTyr577Phe/Ser585Gly mutant was due, at least in part, to the inability of this receptor mutant to transduce pro-survival signals in response to GM-CSF.

It is also noteworthy that while the βcF8 mutant was unable to promote colony formation in response to GM-CSF (FIG. 2), it was not defective in mediating cell survival (FIG. 3). This would imply that while tyrosine phosphorylation of βc is not important for regulating hemopoietic cell survival in response to GM-CSF, it may be important in regulating other signalling pathways necessary for colony formation. In addition, we have also consistently observed that the βcTyr577Phe mutant promotes increased survival even in the absence of GM-CSF. This data, together with findings in Example 1 that the βcTyr577Phe mutant promotes increased colony number (FIG. 2) and colony size would suggest that Tyr577 of βc plays an important role in negatively regulating GM-CSF signalling and that these signals are clearly distinct and dissectable from those generated by the Tyr577/Ser585 bidentate motif.

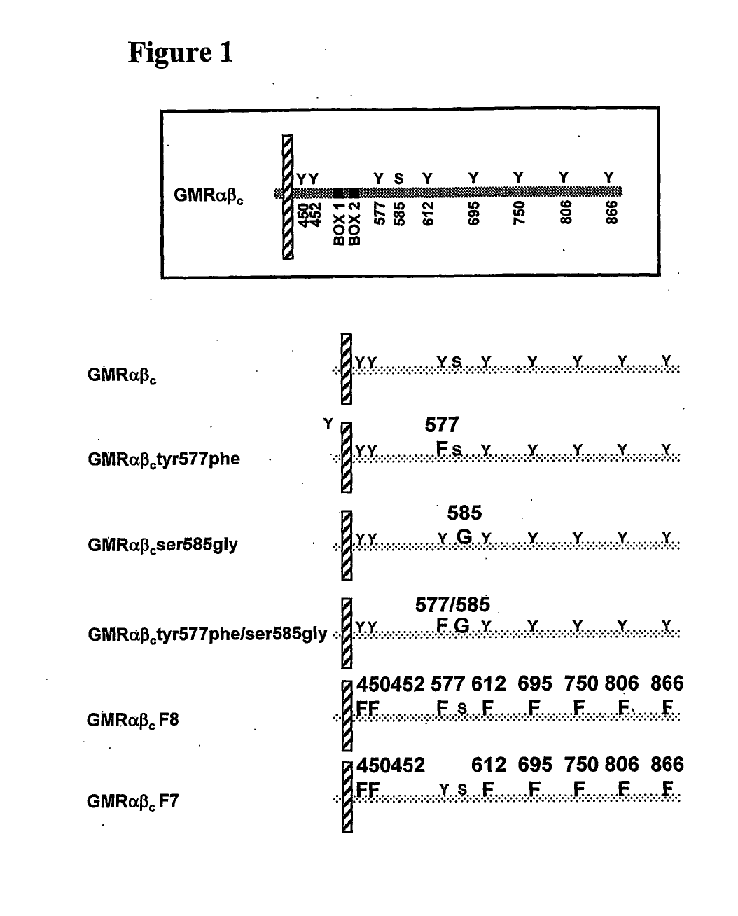

To examine the role of Ser585 and βc tyrosine residues in the ability of GM-CSF to regulate primary hemopoietic cell function we have generated a panel of bicistronic retroviral vectors that allow the co-expression of the human GM-CSF α subunit (GMRα) and either wild type (wt) or mutant common β subunit (βc) in primary mouse hemopoietic cells derived from fetal livers. This panel of βc mutants includes a Ser585Gly mutant which we have shown to be defective in 14-3-3 binding (pRUF-IRES-GMRαβcSer585Gly), a Tyr577Phe mutant which is shown to be defective in Shc binding (pRUF-IRES-GMRαβcTyr577Phe), a Ser585Gly/Tyr577Phe double mutant (pRUF-IRES-GMRαβcSer585Gly/Tyr577Phe), a mutant in which all 8 βc cytoplasmic tyrosines were substituted for phenylalanine (pRUF-IRES-GMRαβcF8), and an add back mutant in which Tyr577 and Ser585 remain intact while all 7 remaining cytoplasmic tyrosines were substituted for phenylalanine (pRUF-IRES-GMRαβcF7). For simplicity, these constructs will be referred to as wtβc, βcTyr577Phe, βcSer585Gly, βcTyr577/Ser585, βcF8 and βcF7 respectively (FIG. 1).

These retroviral constructs were transduced into primary fetal liver cells derived from βc −/− βIL-3 −/− double knockout mice. The ability of the wt and mutant βc to promote colony formation in response to GM-CSF was examined. Transduced fetal liver cells were plated in soft agar in the presence of either GM-CSF or a positive control cytokine cocktail containing stem cell factor (SCF), IL-6 and G-CSF (control cocktail). After 2 weeks, colonies were counted and the results are shown in FIG. 2. GM-CSF was able to promote colony formation in fetal liver cells transduced with the wtβc, βcTyr577Phe, βcSer585Gly and also the βcF7 add back mutant in which Tyr577 and Ser585 remain intact. However, colony formation in response to GM-CSF was essentially abolished in cells expressing either the βcF8 or the βcTyr577Phe/Ser585Gly mutants.

The inability of the βcTyr577Phe/Ser585Gly mutant to promote GM-CSF-mediated colony formation in FIG. 2 indicates that these residues play an essential role in this biological response and that signals generated by Tyr577 and Ser585 cannot be compensated for by any of the remaining 7 βc tyrosine residues. Furthermore, these results define a minimal βc bidentate motif composed of Tyr577 and Ser585 that is not only necessary for promoting GM-CSF-mediated colony formation but is also sufficient for mediating this response as Tyr577 and Ser585 which remain intact in the βcF7 mutant were able to promote colony formation in response to GM-CSF to levels essentially equivalent to that observed for the wtβc.

During the course of these experiments it was consistently observed that not only did the βcTyr577Phe mutant promote increased colony number in response to GM-CSF when compared to cells expressing the wt βc (FIG. 2A), but also colony size was significantly increased. These results suggest that Tyr577 and Ser585 may not only constitute a bidentate motif that is important for promoting colony formation, but that Tyr577 may also have an additional role independent of Ser585 that negatively regulates βc function. We considered the possibility that cells transduced with the βcTyr577Phe mutant were defective in a critical checkpoint in the GM-CSF-mediated differentiation program and that a delay in cellular senescence that normally accompanies terminal differentiation allowed these cells to continue proliferating leading to larger colonies. We therefore examined the hemopoietic cell lineage of the colonies obtained in response to GM-CSF from cells transduced with the wt and mutant βc using an in situ *tristain*. GM-CSF promoted almost exclusively monocyte/macrophage colonies in fetal liver cells transduced with the wtβc. No significant differences between the wtβc and the βcTyr577Phe mutant in terms of their ability to promote the formation of monocyte/macrophage colonies in response to GM-CSF was observed. Similarly, GM-CSF was able to promote monocyte/macrophage colony formation in fetal liver cells transduced with the βcTyr577Phe, βcSer585Gly and the βcF7 mutants indicating that none of these βc mutants were significantly affected in terms of differentiation. Furthermore, no difference in the ability of GM-CSF to upregulate the expression of the F4/80 monocyte/macrophage marker in cells expressing either the wtβc or βcTyr577Phe mutant was observed (data not shown). Thus, the increased colony formation observed for fetal liver cells transduced with the βcTyr577Phe was not due to an overt defect or delay in the differentiation program induced by GM-CSF.

### Example 2

### The Ability of the Wt and Mutant βc to Specifically Promote Cell Survival or Proliferation in Response to GM-CSF

a) Annexin V Staining

The ability of GM-CSF to promote cell survival was determined by annexin V-FLUOS staining essentially as recommended by the manufacturer. Transduced fetal liver cells were plated in IMDM and either 0.1% or 10% HI FCS, containing either no GM-CSF, 50 ng/ml hGM-CSF or 1:1000 pro-survival cocktail (IL-6/EPO/G-CSF). After 48 hours, cells were harvested and stained firstly with the 4H1 anti-GMRα monoclonal antibody and an anti-mouse IgG-PE antibody and secondly with annexin V-FLUOS. Viable cells (annexin V-FLUOS-negative) expressing the GM-CSF receptor (GMRα-positive) were analysed by flow cytometry.

b) Colony Assays

The response of immature cells to GM-CSF was analysed using colony formation assays. Fetal liver cells were transduced and expression of the GM-CSF receptor quantitated as described above. Colony forming cells were assayed using a double layer agar assay. Plates were prepared with underlayers comprised of IMDM supplemented 0.5% agar (Difco) containing cytokines as shown in the figures. An overlayer was added with transduced cells, at a concentration of 100,000 per dish, in 0.3% agar. All media contained 10% HI FCS (JRH Biosciences) with penicillin and streptomycin added.

All cytokines were diluted in PBS and a control of PBS alone was added in each assay to verify that colonies were formed in response to cytokine stimulation. Additionally, all assays included a general stimulus composed of a cocktail of IL-6 (100 ng/ml), SCF (100 ng/ml) and erythropoietin (4 U/ml).

Plates were incubated at 37° C. 5% CO₂ for 14 days and colonies counted using an inverted microscope at the completion of this time.

### Example 3

### The Ability of GM-CSF to Promote the Survival of Fetal Liver Cells Transduced with the βcSer585Gly Mutant Under Low Serum Conditions

While our previous studies had demonstrated a defect in GM-CSF-mediated survival for the βcSer585Gly mutant in the CTL-EN cell line, data presented here indicates that this mutant was not defective in promoting either colony formation (FIG. 2) or survival (FIG. 3A) of primary hemopoietic cells in response to GM-CSF. However, the experiments presented here with fetal liver cells were performed in the presence of 10% FCS which we have previously observed to mask the survival defects of some β_{c} mutants. Fetal liver cells expressing either wtβc or βcSer585Gly were plated out in 0.1% FCS and either no factor, GM-CSF or control cocktail. Under these conditions, GM-CSF was able to promote the survival of fetal liver cells expressing the wtβ_{c} but not the βcSer585Gly mutant (FIG. 3A). These results would indicate that the Ser585 survival pathway that was initially identified in CTL-EN cell lines is also important for regulating the survival of primary hemopoietic cells.

### Example 4

### The Ability of GM-GSA to Promote the Proliferation of Fetal Liver Cells Transduced with Either Wt or Mutant βc Receptors

(a) 5-Bromo-2′-Deoxyuridine (BrdU) Incorporation Assays

The ability of GM-CSF to promote cell cycle progression was determined by BrdU incorporation using the in situ cell proliferation kit (Roche). Briefly, fetal liver cells transduced with either the wt or mutant GM-CSF receptors were firstly stained with the 4H1 anti-GMRα monoclonal antibody followed by anti-mouse IgG-PE and GM-CSF receptor-positive cells were purified using fluoresence activated cell sorting (FACS). Purified cells were then plated out in IMDM and 10% HI FCS containing either no GM-CSF, 50 ng/ml hGM-CSF or control cocktail for 24 hr with the cells being pulsed with BrdU (Roche) for the last four hours. Cells were then fixed and stained with anti-BrdU-FITC antibodies and BrdU incorporation was assessed by flow cytometry.

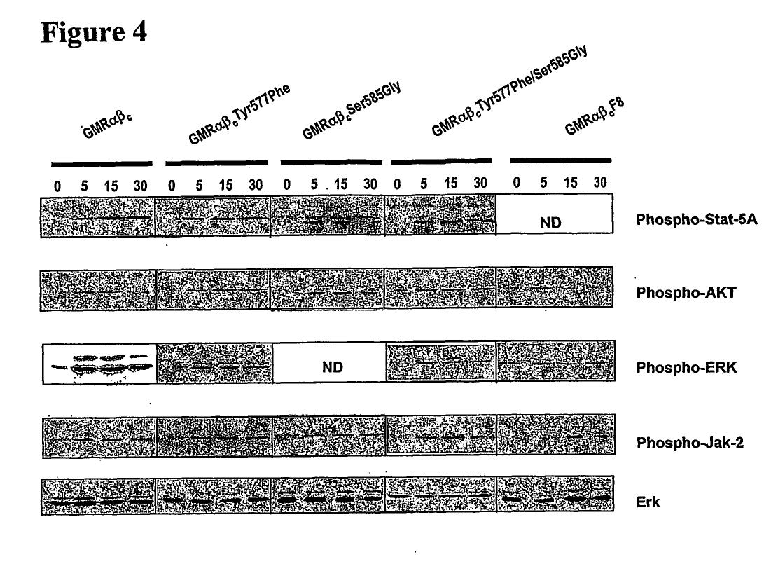

Transduced cells were stained with the 4H1 anti-GMRα monoclonal antibody and an anti-mouse-PE secondary antibody and the GM-CSF receptor-positive cells were purified by FACS. Purified cells were then plated in either GM-CSF or control cocktail for 24 hours with a BrdU pulse for the last 4 hours. Cells were then fixed and analysed for BrdU incorporation by flow cytometry. As shown in FIG. 4, GM-CSF is able to promote BrdU incorporation in cells transduced with the wtβc, βcTyr577Phe, βcSer585Gly, βcTyr577Phe/Ser585Gly, βcF7 but not the βcF8 mutant. Thus, βc tyrosine phosphorylation in response to GM-CSF appears to have an important role in promoting cell proliferation. Furthermore, the results indicate that GM-CSF is able to independently regulate cell proliferation and survival in primary hemopoietic cells and that these biological responses are regulated by distinct motifs in the βc with the βcTyr577Phe/Ser585Gly mutant being unable to regulate cell survival but is able to regulate cell proliferation and the converse being true for the βcF8 mutant which is able to promote cell survival but unable to regulate cell proliferation.

### Example 5

### Ability of the Wt and Mutant βc to Transduce Signals Through the JAK/STAT, the PI 3-Kinase and Ras/Map Kinase Pathways

(a) Immunoblotting

Transduced fetal liver cells were factor-deprived for 12 hours in IMDM containing 0.5% HI FCS and then stimulated with 50 ng/ml human GM-CSF. Cells were lysed in RIPA buffer (150 mM NaCl, 1.0% NP40, 0.5% deoxycholate, 0.1% sodium dodecylsulphate (SDS), 50 mM Tris-HCl, pH 7.4). Cell lysates were then subjected to SDS polyacrylamide gel electrophoresis (PAGE) and immunoblot analysis using standard conditions and signals were developed using enhanced chemiluminescence (ECL)(Amersham Pharmacia or West Dura from Pierce).

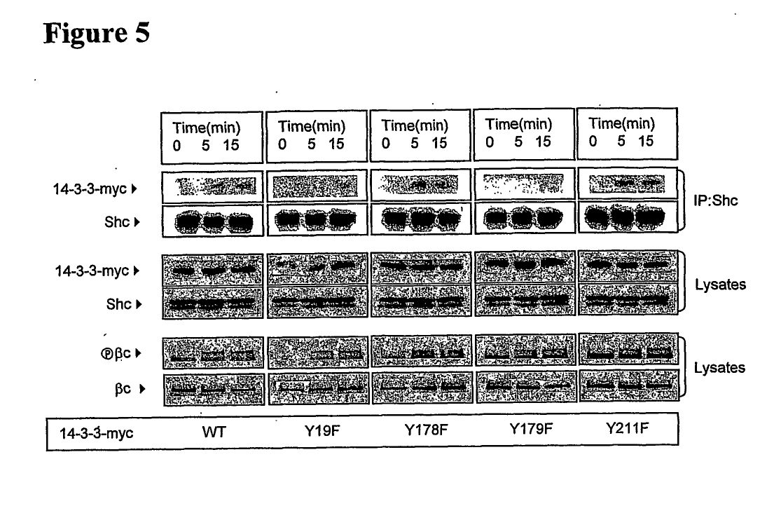

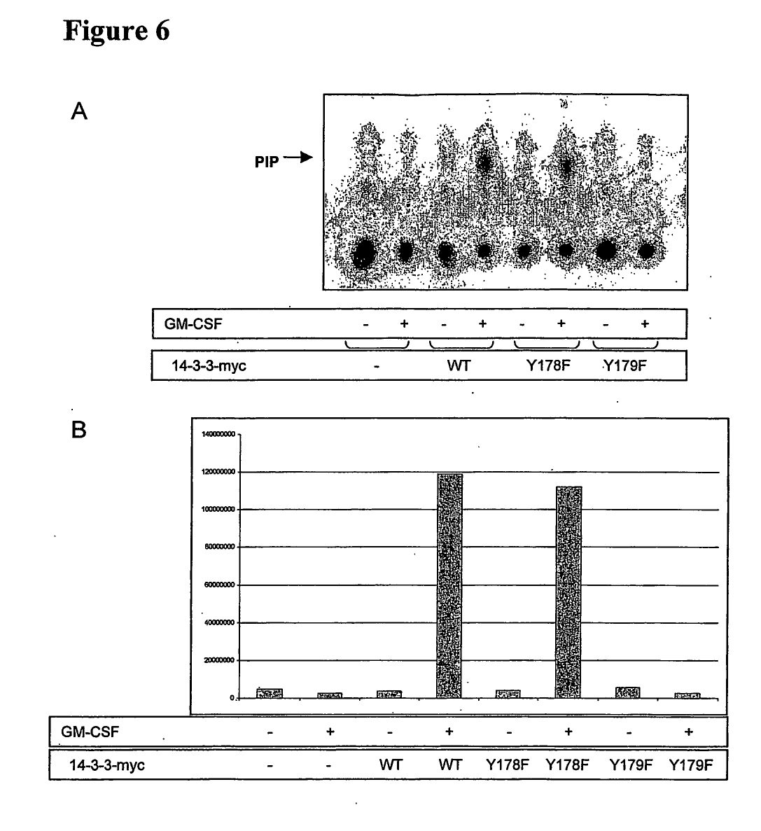

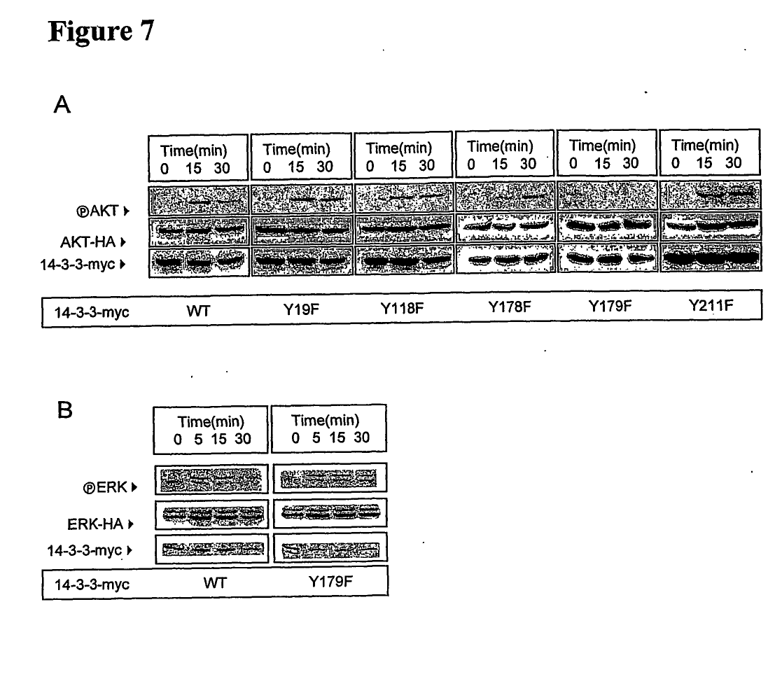

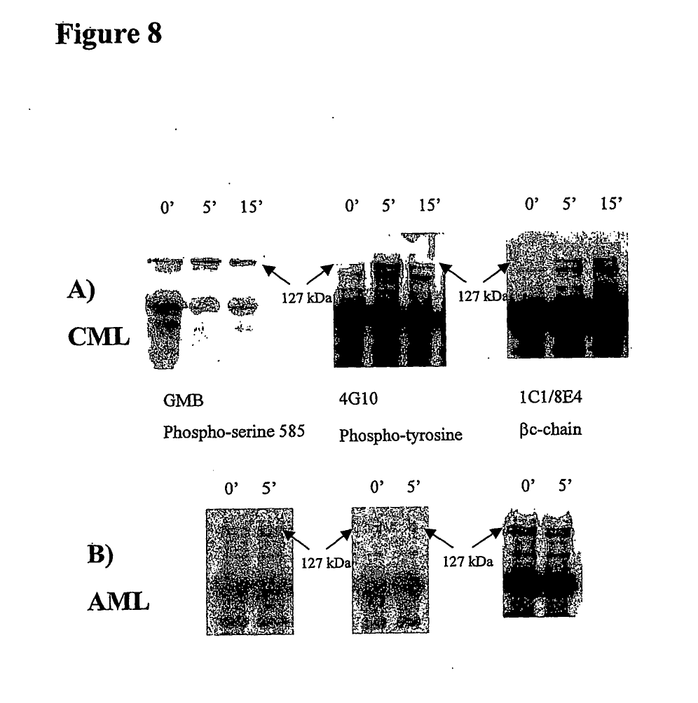

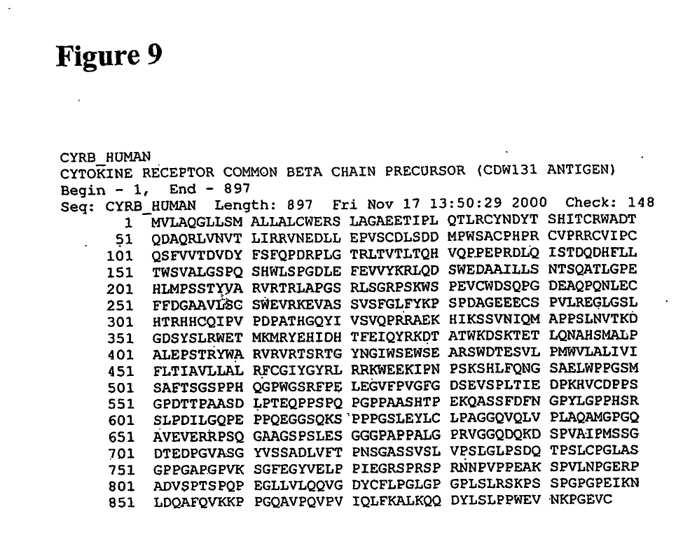

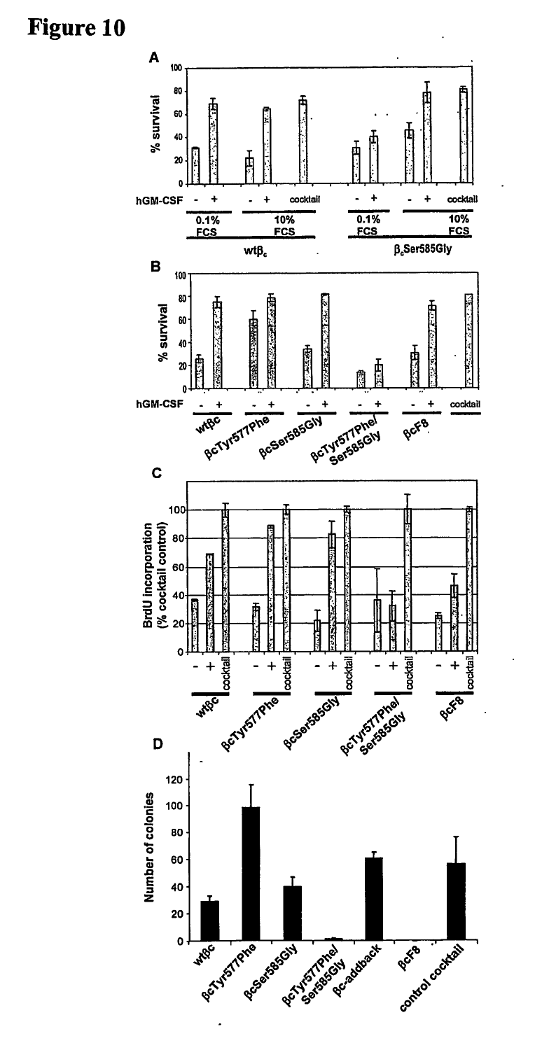

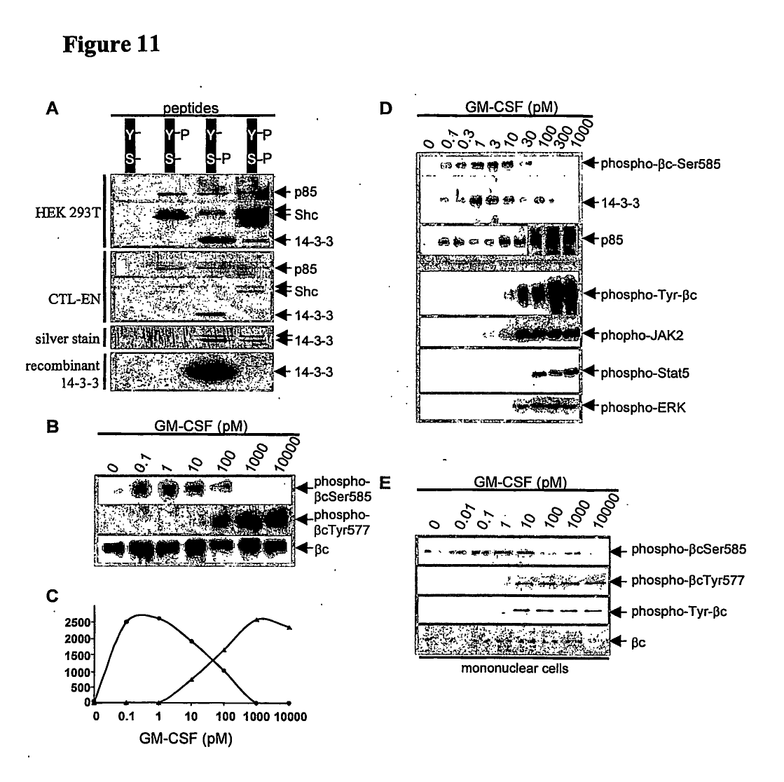

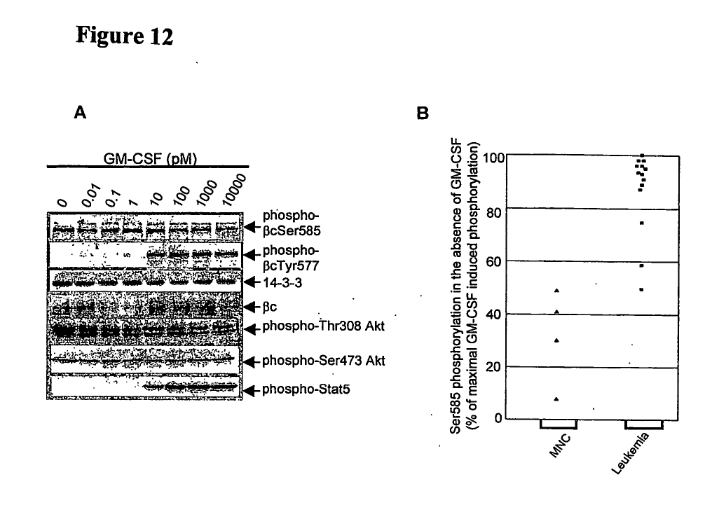

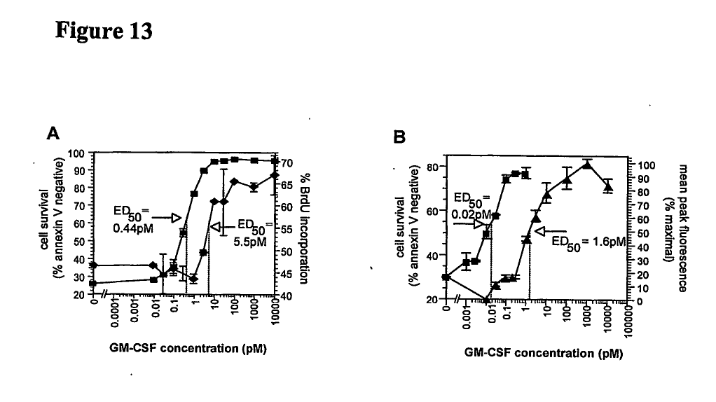

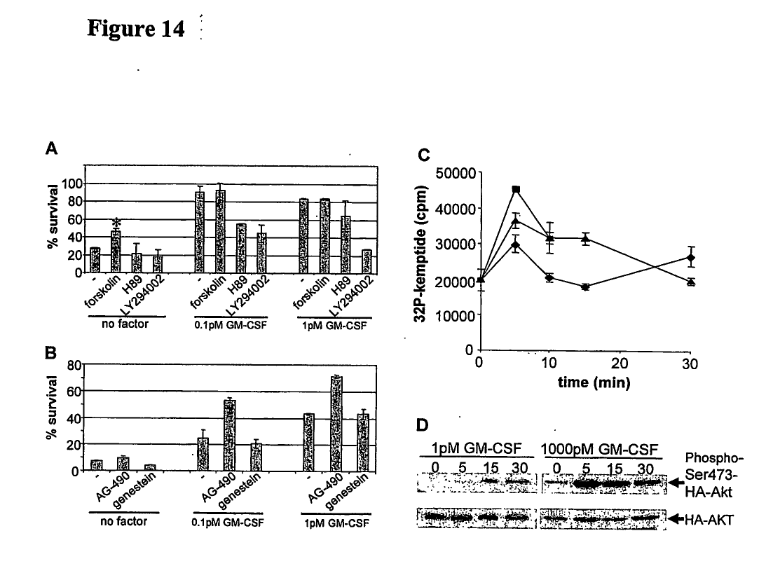

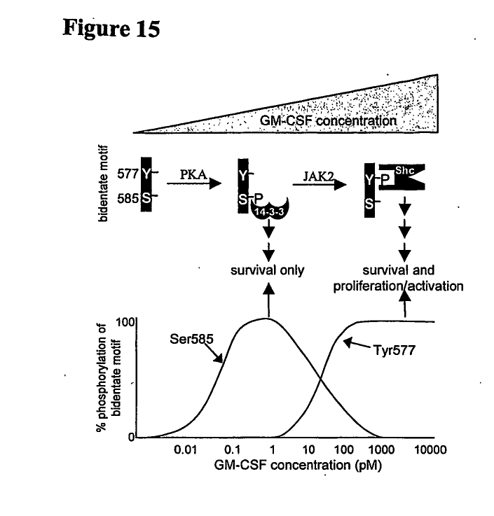

GM-CSF is able to regulate intracellular signalling via the JAK/STAT, the PI 3-kinase and the Ras/Map kinase pathways. To examine the ability of the wt and mutant βc to transduce signals through these pathways a series of Western blots were performed using phospho-specific antibodies to JAK2, STAT5, Akt, ERK Fetal liver cells transduced with the wt βc exhibited increased phosphorylation of JAK2, STAT5, Akt, ERK in response to GM-CSF (FIG. 5). Similarly, cells expressing the βcTyr577Phe, βcSer585Gly, and βcF7 mutants also demonstrated phosphorylation of these signalling molecules. While cells expressing the βcF8 mutant were able to induce phosphorylation of JAK2 and STAT5 in response to GM-CSF, the phosphorylation of Akt and, ERK was decreased compared to that observed in cells expressing the wt βc. The βcTyr577Phe/Ser585Gly mutant, which was unable to promote cell survival in response to GM-CSF, was able to regulate the phosphorylation of JAK2, STAT5, Akt, ERK {and GSK-3b} in a similar manner to that observed for cells expressing the wt βc. Thus the inability of GM-CSF to promote cell survival in fetal liver cells transduced with the βcTyr577Phe/Ser585Gly mutant was not due to an inability of this mutant receptor to regulate the activation of either JAK2, STAT5, Akt, or ERK

### Example 6

### Interactions of the Shc and 14-3-3 to Tyr577 and Ser585 Respectively

To study the possible interaction of Shc, which is known to bind to Tyr 577, and 14-3-3 which we found to bind to Ser 585, we stimulated cells with GM-CSF and measured their association. We found that GM-CSF stimulation caused tyrosine phosphorylation of 14-3-3 and that Tyr 179 was necessary for 14-3-3 interacting with Shc (FIG. 5). Furthermore Tyr 179 of 14-3-3 was also necessary for PI-3 kinase activation (FIG. 6) and AKT activation (FIG. 7) in response to GM-CSF.

The possibility that the bidentate motif or parts of it could be intrinsically activated in leukaemia was also searched. Importantly we found in primary cells from CML or AML patients that intrinsic activation of Ser 585 was taking place in both cases (FIG. 8) indicating that this motif and its abnormal activation may play a role in disease.

### Example 7

### Identification of Similar Potential Bidentate Motifs in Other Receptors

The identification of a novel phosphotyrosine/phosphoserine bidentate motif that is important in regulating cell survival in these studies prompted us to examine whether other cell surface receptors may also contain similar motifs. Phosphorylated Tyr577 of βc binds Shc via its PTB domain whereas phosphorylated Ser585 binds 14-3-3. We therefore scanned the cytoplasmic domains of cell surface receptors for a PTB binding site followed by a 14-3-3 binding site using software available to the skilled addressee. The PTB domain of Shc recognizes a N-X-X-Y motif (where Y is phosphorylated). 14-3-3 was originally demonstrated to binding two possible motifs; a mode 1 site (R-S-X-S/T-X-P) or a mode 2 site (R-X/Ψ-X/Ψ-X/Ψ-S/T-X-P)(where S or T is phosphorylated and Ψ is a hydrophobic residue). Variations on these prototypic 14-3-3 binding motifs have since been reported with K, H or Q also being tolerated at the −3 and −4 positions relative to the phosphoserine/phosphothreonine. In addition, the proline at the +2 position, which has been reported to be important for the correct exit of the bound protein from the binding groove of 14-3-3, has been found to be dispensible if the 14-3-3 binding motif occurs close to the C-terminus of a protein. Searching for motifs that allow these variations, we have identified conserved putative bidentate tyrosine/serine motifs in a range of cell surface receptors (Table 1). In addition to the notable prevalence of such a bidentate motif in cell surface receptors, it is also striking that in some cases this motif appears to be conserved within specific members of receptor families such as the FGF, LDL and integrin receptor families. Alignment of these motifs suggests a putative consensus bidentate motif, N-X-X-Y-(X)₁₋₁₃-[R/K/H/Q]-[X/Ψ]₂₋₃-S/T-X-P (where X is any residue, Y is phosphotyrosine, S/T is phosphoserine or phosphothreonine and Ψ is a hydrophobic residue). We also considered the possibility that receptors may also utilize alternative motifs in which the tyrosine residue was not part of a PTB binding site but rather an SH2 binding site. Searching for an adjacent tyrosine residue/14-3-3 binding site, we identified alternative putative bidentate motifs in a range of cell surface receptors. Alignment of these motifs gave the consensus Y-(X)₁₋₁₆-[R/K/H/Q]-[X/Ψ]₂₋₃-S/T-X-P. Our finding that the Tyr577/Ser585 bidentate motif is important in regulating cell survival in response to GM-CSF and that similar motifs are also found in other cell surface receptors suggests that this novel motif may play a fundamental role in regulating intracellular signalling in response to a wide range of cytokines and growth factors.

TABLE 1
PILE UP:

| | |
|---|---|
| betaR .... | NGPYLG...........PP..HSRSLP |
| Acetylcholine R (ISOFROM?) | NVHYRT...........P...KTHTMP |
| Acetylcholine R alpha-5 | **RYFTQKEE..........TESGSGP |
| (CONSERV?) | |
| C-C chemokine receptor 6 | NKKYELQDRDVCE....P.RYRSVSEP |
| Middle T antigen | NPTYSVM..............RSHSYP |
| integrin alpha 1 | NIFYLIR...KSGSFPMPELKLSISFP |
| FGFR2 (KGF R) | NEEYLDLSQ........PLEQYSPSYP |
| FGFR1 (fig) | NQEYLDLSM........PLDQYSPSFP |
| FGFR5 | NATYKVD...........VIQRTRSKP |
| Erb4 | NPEY................HSASSGP |
| Erb4 (second) | NPDY.................WNHSLP |
| Vaccinia virus protein A36R | NPSYSSNPFVNYN....KTSICSKSNP |
| Macrophage mannose R (MRC1) | NTLY................FNSQSSP |
| LDLR | NPVYQKTTEDEVHI...CHNQDGYSYP |
| VLDL (rat) | NPVYLKTTEEDLSIDIG..RH.SASVG |
| | (near end of protein) |
| LRP1 low density lipoprotein | NPTYKMYEGGEPDDVGGLLDADFALDPDKPTNFTNPVY |
| receptor-related protein 1 | |
| integrin beta 1 | NPIY...........KSAVTTVV |
| | (end of protein) |
| interin beta 7 | NPLY...........KSAITTTV |
| | (end of protein) |
| integrin beta 3 | NPLY...........KEATSTFT |
| | (end of protein) |
| integrin beta 5 | NPLY...........RKPTSTHT |
| | (end of protein) |
| integrin beta 6 | NPLY...........RGSTSTFK |
| G-CSFR1 (second) | PGHYL................RCDSTQP |
| G-CSFR1 | VQTYVLQ...........GDPRAVSTQP |
| g-csf-r | QVLYGQLL.............GSPTSP |
| (CHECK?) | |
| IL-6B (gp130) | HSGYRHQVPSVQVF.....SRSESTQP |
| leptinR. | WKMYEVYDA.........KS.KSVSLP |
| prolactinR... | KIPYFHA..........GGS.KCSTWP |
| insulinR | ELDYCLKGLKL......P.S.RTWSPP |
| irs-1 .... | SGDYMPM............SPKSVSAP |
| IGFI R | SFYYSEENKLPEPEELDLEPENMESVP (LDPSASSSSLP) 1283 = surv1. |
| flt3 R | EEIYIIM.....QSCWAFDSRKRPSFP |
| VEGFR2 (FLK1) | ISQYLQN............S.KRKSRP |
| PDGF R-alpha | GTAY................GLSRSQP |
| IL-9R | ***YLPQEDWAP.........TSLTRP (CONSERV?) |
| p75 NTR | LVAYIAFKRWNSCKQN...KQGANSRPVNQTPPPEGEKLHSDSGIS |
| | (phosphorylated) |
| MOTIF (forward) | n-X-X-Y-X (3, 17) - [RKHQ] -X (2, 3) - [ST] -X-P |
| EGFR | RYSSDPTGALTEDSIDDTFLPVPEYINQSVPKRPAGSVQNPVY.. (NPEY) |
| Erb2 | KTLSPGKNGVVKDVFTF.................GGAVENPEY |
| Voltage-depend | RTHSLP.................................NDSY |
| T-type Ca chan. | |
| alpha-1G subunit | |
| EPO R | SDGPYSNPYENSLIPAAEPLPPSYVACS |
| | (Y NB in PI 3-K; S is end of protein, JBC 270: 23402) |
| MOTIF (reverse) | [RXHQ] -X(2, 3) - [ST] -X-P-X (0, 33) -N-X-X-Y |
| TRHR receptor | HFSTELD |
| IL-2R beta | NQGYFFFHLPDALEIEACQVYFTYDPYSEEDPDEGVAGAPTGSSP |

### Example 8

### Tyr577 and Ser585 Constitute a Bidentate Motif that is Necessary and Sufficient for GM-CSF-Mediated Survival and Proliferation of Primary Hemopoietic Cells

To study the pleiotropic responses of cells to GM-CSF we developed a system in which primary mouse fetal liver cells capable of survival, proliferation and differentiation were used as a source of hemopoietic cells. These were transduced with bicistronic retroviral vectors for the expression of the human GMRα and βc subunits. We used fetal liver cells from βc −/− βIL-3 −/− double-knockout mice to avoid artifacts arising from the interaction of transduced human βc with endogenous mouse β chains. We firstly showed that although Ser585 of βc was essential for GM-CSF-mediated cell survival in 0.1% FCS, neither Ser585 nor Tyr577 individually were essential when 10% FCS was used (FIG. 10A, B). These results suggested that these residues were compensating for each other or that other elements in the receptor were involved in the regulation of survival. We then transduced fetal liver cells with βc mutants carrying the double mutation (βcTyr577Phe/Ser585Gly) or all eight tyrosines mutated to phenylalanine (βcF8). Whilst the βcF8 mutant showed no apparent defect in GM-CSF-mediated cell survival, the βcTyr577Phe/Ser585Gly double mutant was completely unable to promote cell survival (FIG. 10B). We then examined whether the same residues that were important for regulating cell survival were also important for regulating cell proliferation. GM-CSF promoted BrdU incorporation in cells transduced with the wtβc, βcTyr577Phe and βcSer585Gly, however, incorporation was essentially abolished with the βcTyr577Phe/Ser585Gly mutant (FIG. 10C). Furthermore, BrdU incorporation was dramatically reduced in the βcF8 mutant (FIG. 10C).

We next examined the ability of wtβc or Ser585 and Tyr577 mutants to support hemopoietic cell colony formation in an assay that depends on both survival and proliferation. We observed that cells expressing the βcTyr577Phe mutant supported colony formation when compared to cells transduced with the wtβc (FIG. 10D). In fact, the number of colonies were consistently higher than with the wt receptor suggesting that Tyr577 also plays a role in restraining βc activity both in the presence and absence of GM-CSF. No defect in colony formation was observed in cells transduced with the βcSer585Gly mutant, however, GM-CSF could not promote colony formation in cells transduced with the βcTyr577Phe/Ser585Gly double mutant. Importantly, no defect in colony formation was observed in cells expressing the βc-addback mutant in which Ser585 and Tyr577 remain intact but all remaining tyrosines were substituted for phenylalanine. Together, these results suggest that Tyr577 and Ser585 are both necessary and sufficient for regulating hemopoietic cell survival and proliferation and as such constitute a structurally and functionally identifiable bidentate motif. In addition, fetal liver cells transduced with the βcF8 mutant which were able to survive (FIG. 10B) but not proliferate (FIG. 10C) in response to GM-CSF, did not form colonies in response to GM-CSF (FIG. 10D).

### Example 9

### Tyr577 and Ser585 in the Bidentate Motif Couple to Alternate Signalling Complexes

One interpretation of these results is that Tyr577 and Ser585 regulate redundant signalling pathways that promote survival and proliferation and that Tyr577 and Ser585 can compensate for each other. In this scenario 14-3-3 may bind Ser585 and Shc may bind Tyr577 simultaneously to regulate redundant signalling pathways and that it is only following mutation of both residues that a functional defect is revealed. Such a signalling mechanism assumes that there is no steric hindrance between 14-3-3 and Shc binding to the βc (Tyr577 and Ser585 lie 8 residues apart). To test this possibility we performed pulldown experiments to examine the ability of 14-3-3, Shc and p85 from cell lysates derived from HEK-293-T and CTL-EN cells to bind phospho-peptides encompassing Tyr577 and Ser585 of βc. The non-phospho-Tyr577/non-phospho-Ser585 control peptide did not precipitate p85, Shc or 14-3-3 from HEK-293T or CTL-EN lysates (FIG. 2A lane 1). The phospho-Tyr577/non-phospho-Ser585 peptide precipitated p85 and Shc but not 14-3-3 (FIG. 11A lane 2). On the other hand, the non-phospho-Tyr577/phospho-Ser585 peptide precipitated p85 and 14-3-3 (FIG. 11A lane 3). Notably, while the phospho-Tyr577/phospho-Ser585 doubly phosphorylated peptide precipitated p85 and Shc (FIG. 11A lane 4), the binding of 14-3-3 was markedly reduced. To examine the possibility that specific isoforms of 14-3-3 that were not recognized by our anti-14-3-3 antibodies were able to bind the phospho-Tyr577/phospho-Ser585 peptide, we subjected pulldowns to SDS-PAGE and silver-staining. While additional isoforms were detected using this approach, the overall binding of 14-3-3 proteins to the phospho-Tyr577/phospho-Ser585 peptide was reduced compared to the non-phosphoTyr577/phospho-Ser585 peptide (FIG. 11A, silver stain). While purified recombinant 14-3-3 was able to bind the non-phospho-Tyr577/phospho-Ser585 peptide in a cell-free system, no binding to the phospho-Tyr577/phospho-Ser585 doubly phosphorylated peptide was detected, even with long exposures (FIG. 11A, recombinant 14-3-3). Thus our results indicate that 14-3-3 is unable to bind phospho-Ser585 when Tyr577 is phosphorylated and that the binding of 14-3-3 to Ser585 and the binding of Shc to Tyr577 occur in a mutually exclusive manner and may thus lead to the activation of alternative signalling pathways. Furthermore, the ability of peptides to recruit p85 regardless of whether they were phosphorylated on Tyr577 or Ser585 suggests that βc may utilize alternate modes of recruitment of PI 3-kinase.

### Example 10

### Tyr577 and Ser585 are Independently and Differentially Phosphorylated and Function as a Binary Switch that is Regulated by GM-CSF Concentration

To directly investigate the possibility that Tyr577 and Ser585 regulate alternative signalling pathways, we examined the phosphorylation of Tyr577 and Ser585 in response to GM-CSF. We developed phospho-specific antibodies (Guthridge et al., 2000; Guthridge et al., 2004) that showed that GM-CSF rapidly induced the phosphorylation of Ser585 and Tyr577 with similar kinetics (data not shown). Importantly however, clear differences in the regulation of Ser585 and Tyr577 phosphorylation following titration of GM-CSF were observed. Phosphorylation of Ser585 increased in response to <3 pM GM-CSF, decreased at 10 pM and was virtually absent over 100 pM (FIG. 11B,C). In contrast, Tyr577 phosphorylation was detectable only at GM-CSF concentrations greater than 10 pM and remained elevated (FIG. 11B,C).

This differential phosphorylation of the GM-CSF receptor was further studied by examining the activation of key signalling pathways in response to GM-CSF. We found that in addition to the detection of Ser585 phosphorylation, 14-3-3 recruitment was also observed at low GM-CSF concentrations (FIG. 11D). Although no βc tyrosine phosphorylation was detected within this concentration range, p85 recruitment was also observed. Higher concentrations of GM-CSF (>10 pM) that were able to promote βc tyrosine phosphorylation also resulted in p85 recruitment as well as the phosphorylation of JAK2, STAT5 and ERK. Furthermore, a similar switch mechanism in Ser585 and Tyr577 phosphorylation was also observed in primary peripheral blood mononuclear cells (FIG. 11E). Thus, while Ser585 and Tyr577 are essential for the regulation of hemopoietic cell survival and proliferation (FIG. 10), these residues do not appear to regulate redundant signalling pathways. In fact, the results shown in FIG. 11 suggest that βc is able to generate two distinct and qualitatively different types of signals. One at low GM-CSF concentrations (<3 pM) that promotes Ser585 phosphorylation, 14-3-3 binding and p85 recruitment and the other at higher concentrations of cytokine (>10 pM) that promotes Tyr577 phosphorylation and the activation of the JAK/STAT and Ras/MAP kinase pathways. Thus, Ser585 and Tyr577 appear to function as a binary switch that is regulated by GM-CSF concentrations where either Ser585 or Tyr577 is phosphorylated.

### Example 11

### The Binary Switch Function of Tyr577 and Ser585 Becomes Deregulated in Some Myeloid Leukemias

Our findings show that the Tyr577/Ser585 bidentate motif is necessary and sufficient for the regulation of hemopoietic cell survival and proliferation (FIG. 10). Deregulated survival and proliferation are classical hallmarks of cancer. We therefore examined the phosphorylation of Tyr577 and Ser585 in mononuclear cells derived from patients with myeloid leukemia. In contrast to the results obtained in primary mononuclear cells obtained from normal donors (FIG. 11E), we observed in cells derived from a patient with AML that Ser585 phosphorylation, Akt phosphorylation and recruitment of 14-3-3 were constitutive while Tyr577 phosphorylation and the phosphorylation of STAT5 were normally regulated by cytokine (FIG. 12A). We have observed a similar pattern of deregulated Ser585 phosphorylation in 11/13 leukemias tested so far including AML (6/7), CML (3/4), CMML (2/2) and in a case of myelofibrosis (FIG. 12B). Such a signalling defect raises the possibility that a survival advantage may be conferred to leukemic cells by constitutive Ser585 phosphorylation.

### Example 12

### Tyr577 and Ser585 Couple to Different Biological Outcomes

To determine how the signalling pathways emanating from Tyr577 and Ser585 relate to the ability of GM-CSF to promote biological responses, we performed dose-response analysis of GM-CSF-mediated cell survival and proliferation in the TF-1 cell line and also cell survival and activation (or “priming”) in human neutrophils. The calculated ED₅₀ for TF-1 survival was 0.44 pM while the ED₅₀ for cell proliferation was 5.5 pM (FIG. 13A). Consistent with earlier reports (Begley et al., 1986), the calculated ED₅₀ for survival in primary human neutrophils was 0.02 pM while the ED₅₀ for the upregulation of the activation marker CD11b (α-chain of MAC-1) was 1.6 pM (FIG. 13B). These findings reveal an important relationship between the regulation of pleiotropic biological responses by GM-CSF: concentrations of GM-CSF able to promote maximal Ser585 phosphorylation (<3 μM) were also able to promote maximal survival in TF-1 cells. Importantly, these concentrations of cytokine can promote cell survival in the absence of detectable βc tyrosine phosphorylation or cell proliferation (FIGS. 11 and 13A). As the concentration of GM-CSF is increased over 10 pM, a switch in signalling occurs and the phosphorylation of Ser585 decreases, the phosphorylation of Tyr577 increases, activation of the JAK/STAT and Ras/MAPK pathways occurs and TF-1 cells undergo proliferation as well as survival (FIGS. 11 and 13A). Similarly, the ED₅₀ for the survival of primary human neutrophils in response to GM-CSF was significantly lower than the ED₅₀ for activation. Together, these results suggest that not only do Tyr577 and Ser585 function as a binary switch for the activation of either the Shc or the 14-3-3 signalling pathways respectively, but that this switch is functionally linked to the regulation of distinct and independent biological responses.

### Example 13

### Hemopoietic Cell Survival by Low GM-CSF Concentrations is Regulated Via a Phosphotyrosine-Independent PKA and PI 3-Kinase Pathway

As our results suggest that low concentrations of GM-CSF (<3 pM) activate a novel signalling pathway that results in Ser585 phosphorylation and the selective regulation of cell survival in the absence of detectable βc tyrosine phosphorylation, we examined the possible mechanisms involved. A potential candidate is PKA as we previously showed that PKA phosphorylates Ser585 in vitro (Guthridge et al., 2000). We found that activation of PKA by forskolin in neutrophils in the absence of GM-CSF significantly increased cell survival from 27% to 46% (*p-value <0.02), although this level of cell survival was less than that promoted by either 0.1 pM GM-CSF (90%) or 1 pM GM-CSF (83%)(FIG. 14A). Similar results were obtained with forskolin in TF-1 cells (data not shown). Pharmacological blockade of PKA activity using H89 reduced the ability of GM-CSF to promote survival in both TF-1 cells (data not shown) and neutrophils (FIG. 14A). PI 3-kinase is an important regulator of cell survival and phosphotyrosine signalling pathways are thought to be important for its activation. However, our results show that inhibition of PI 3-kinase with LY294002 blocks survival of neutrophils mediated by concentrations of GM-CSF that do not promote detectable βc tyrosine phosphorylation (FIG. 14A). To further assess the role of tyrosine phosphorylation signalling pathways in GM-CSF-mediated survival, we examined the effects of genestein or a specific JAK2 inhibitor (AG490). Neither inhibitor was able to block neutrophil survival in response to low concentrations of GM-CSF (FIG. 14B). In fact survival was increased in the presence of AG490. These results would suggest that low concentrations of GM-CSF (1 pM) are able to promote survival via PKA and PI 3-kinase signalling in the absence of tyrosine phosphorylation. We therefore examined the regulation of PKA activity and the phosphorylation of Akt in response to 1 pM and 1000 pM GM-CSF. We found that 1 pM GM-CSF was able to activate PKA activity in TF-1 cells and that this response was higher and more sustained than that observed for 1000 pM (FIG. 14C). We were also able to detect the phosphorylation of Ser473 of Akt in response to 1 pM GM-CSF, although the magnitude was reduced and the kinetics were delayed in comparison to 1000 pM GM-CSF (FIG. 14D). Thus our results show that 1 pM GM-CSF is able to activate a signalling pathway that leads to PKA activation, Akt phosphorylation and cell survival, and that phosphotyrosine signalling pathways are not required for these events.

### REFERENCES

Begley, C. G., Lopez, A. F., Nicola, N. A., Warren, D. J., Vadas, M. A., Sanderson, C. J., and Metcalf, D. (1986). Purified colony-stimulating factors enhance the survival of human neutrophils and eosinophils in vitro: a rapid and sensitive microassay for colony-stimulating factors. Blood 68, 162-166.
Guthridge, M. A., Stomski, F. C., Barry, E. F., Winnall, W., Woodcock, J. M., McClure, B. J., Dottore, M., Berndt, M. C., and Lopez, A. F. (2000). Site-specific serine phosphorylation of the IL-3 receptor is required for hemopoietic cell survival. Mol. Cell 6, 99-108.
Guthridge, M. A., Stomski, F. C., Thomas, D., Woodcock, J. M., Bagley, C. J., Berndt, M. C., and Lopez, A. L. (1998). Mechanism of activation of the GM-CSF, IL-3 and IL-5 family of receptors. Stem Cells 16, 301-313.
Le, F., Stomski, F., Woodcock, J., Lopez, A. F., and Gonda, T. J. (2000). The role of disulfide-linked dimerization in interleukin-3 receptor signalling and biological activity. J Biol Chem 275, 5124-5130.
McClure, B., Stomski, F., Lopez, A., and Woodcock, J. (2001). Perverted responses of the human granulocyte-macrophage colony-stimulating factor receptor in mouse cell lines due to cross-species beta subunit association. Blood 98, 3165-3168.
Peters, S. O., Kittler, E. L., Ramshaw, H. S., and Quesenberry, P. J. (1996). Ex vivo expansion of murine marrow cells with interleukin-3 (IL-3), IL-6, IL-11, and stem cell factor leads to impaired engraftment in irradiated hosts. Blood 87, 30-37.
Robb, L., Drinkwater, C. C., Metcalf, D., Li, R., Kontgen, F., Nicola, N. A., and Begley, C. G. (1995). Hematopoietic and lung abnormalities in mice with a null mutation of the common β subunit of the receptors for granulocyte-macrophage colony-stimulating factor and interleukins 3 and 5. Proc. Natl. Acad. Sci. (USA) 92, 9565-9569.
Stomski, F. C., Dottore, M., Winnall, W., Guthridge, M. A., Woodcock, J., Bagley, C. J., Thomas, D. T., Andrew, R. K., Berndt, M. C., and Lopez, A. F. (1999). Identification of a 14-3-3 binding sequence in the common p chain of the GM-CSF, IL-3 and IL-5 receptor that is serine-phosphorylated by GM-CSF. Blood 94, 1933-1942.

Finally, it is to be understood that various other modifications and/or alterations may be made without departing from the spirit of the present invention as outlined herein.

1-79. (canceled)

80. A bidentate motif capable of binding a cytoplasmic protein and activating cellular activities in a cell, said bidentate motif comprising a tyrosine and a serine/threonine residue which are capable of interaction with cytoplasmic proteins, and wherein the residue and cytoplasmic protein can interact to activate cellular activity in the cell.

81. A bidentate motif according to claim 80 wherein the tyrosine and serine/threonine residue comprises a binary switch for independent regulation of cellular activity.

82. A bidentate motif capable of binding to a cytoplasmic protein according to claim 80 comprising a tyrosine and a serine/threonine residue, said motif comprising an amino acid sequence alignment selected from the group consisting of:
N-X-X-Y-(X)₁₋₁₃-[R/K/H/Q]-[X/Ψ]₂₋₃-S/T-X-P (SEQ ID NO: 71)
wherein X is any residue, Y is tyrosine, S/T is serine or threonine and Ψ is a hydrophobic residue or an equivalent thereof; or
Y-(X)₁₋₁₆-[R/K/H/Q]-[X/Ψ]₂₋₃-S/T-X-P (SEQ ID NO: 72)
wherein X is any residue, Y is tyrosine, S/T is serine or threonine and Ψ is a hydrophobic residue or an equivalent thereof; or
N-X-X-Y-[X]₁₋₃₀-[R/K/Q′H]-[X]₁₋₄-[S/T]-X-p (SEQ ID NO: 73)
wherein X is any residue, Y is phosphotyrosine, S/T is phosphoserine/phosphothreonine.

83. A bidentate motif according to claim 80 wherein the motif is derived from a receptor.

84. A bidentate motif according to claim 80 wherein the motif is derived from the common beta chain (βc).

85. A bidentate motif according to claim 80 wherein the tyrosine is equivalent to Tyr577 of the common beta chain (βc) and/or the serine is equivalent to Ser 585 of the common beta chain (βc).

86. A bidentate motif according to claim 80 wherein the tyrosine or serine/threonine are independently phosphorylated in response to a cytokine, and phosphorylation is dependent on the cytokine concentration.

87. A bidentate motif according to claim 80 wherein phosphorylation of the serine independently of the tyrosine regulates cell survival.

88. A bidentate motif according to claim 80 wherein phosphorylation of the tyrosine independent of the serine regulates cell survival and proliferation.

89. A bidentate motif according to claim 83, with a modification at a residue equivalent to the Tyr 577 and/or Ser585.

90. The bidentate motif according to claim 89 wherein the residue equivalent to Tyr 577 is substituted with phenylalanine and/or the Ser 585 residue is substituted with glycine.

91. A method of modulating cellular activity in a cell, said method comprising: modulating phosphorylation of a tyrosine and/or serine residue of a bidentate motif capable of binding to a cytoplasmic protein comprising a tyrosine and a serinelthreonine residue, said motif comprising an amino acid sequence alignment selected from the group consisting of:
N-X-X-Y-(X)₁₋₁₃-[R/K/H/Q]-[X/Ψ]₂₋₃-S/T-X-P (SEQ ID NO: 71)
wherein X is any residue, Y is tyrosine, S/T is serine or threonine and Ψ is a hydrophobic residue or an equivalent thereof; or
Y-(X)₁₋₁₆-[R/K/H/Q)-[X/Ψ]₂₋₃-S/T-X-P (SEQ ID NO: 72)
wherein X is any residue, Y is tyrosine, S/T is serine or threonine and Ψ is a hydrophobic residue or an equivalent thereof; or
N-X-X-Y-[X]₁₋₃₀-[R/K/Q/H]-[X]₁₋₄[S/T]-X-p (SEQ ID NO: 73)
wherein X is any residue, Y is phosphotyrosine, S/T is phosphoserine/phosphothreonine.

92. A method according to claim 91 wherein the phosphorylation is modulated by mutating the tyrosine and/or serine.

93. A method according to claim 92 wherein the tyrosine is substituted for phenylalanine and/or the serine is substituted for glycine.

94. A method according to claim 91 wherein the phosphorylation is decreased by subjecting the cell to an antagonist or kinase inhibitor which inhibits phosphorylation of the tyrosine and/or serine.

95. A method according to claim 91 wherein cellular activity is inhibited, said method comprising decreasing or inhibiting phosphorylation of the tyrosine and/or serine of the bidentate motif.

96. A method according to claim 95 wherein the cellular activity is cell survival, said method comprising inhibiting phosphorylation of the serine.

97. A method according to claim 95 wherein the cellular activity is cell survival, said method comprising inhibiting phosphorylation of the serine equivalent to Ser585 of the common βc.

98. A method according to claim 91 wherein cellular activity is activated, said method comprising inducing phosphorylation of the tyrosine and/or serine of the bidentate motif.

99. A method according to claim 98 wherein the cellular activity is cell survival, said method comprising increasing phosphorylation of the serine.

100. A method according to claim 91 wherein the cellular activity is cell proliferation, said method comprising increasing phosphorylation of the tyrosine.

101. A method of treating a cytokine mediated condition, said method comprising:
regulating activation of phosphorylation of a tyrosine and/or serine of a bidentate motif capable of binding to a cytoplasmic protein comprising a tyrosine and a serine/threonine residue, said motif comprising an amino acid sequence alignment selected from the group consisting of:
N-X-X-Y-(X)₁₋₁₃-[R/K/H/Q]-[X/Ψ]₂₋₃-S/T-X-P (SEQ ID NO: 71)
wherein X is any residue, Y is tyrosine, S/T is serine or threonine and Ψ is a hydrophobic residue or an equivalent thereof; or
Y-(X)₁₋₁₆-[R/K/H/Q]-[X/Ψ]₂₋₃-[S/T]-X-P (SEQ ID NO: 72)
wherein X is any residue, Y is tyrosine, S/T is serine or threonine and Ψ is a hydrophobic residue or an equivalent thereof; or
N-X-X-Y-[X]₁₋₃₀-[R/K/Q/H]-[X]₁₋₄-[S/T]-X-p (SEQ ID NO: 73)
wherein X is any residue, Y is phosphotyrosine, S/T is phosphoserine/phosphothreonine.

102. A method according to claim 101 wherein the cytokine mediated condition is treated by increasing or decreasing activation of phosphorylation of the tyrosine and/or serine of the bidentate motif.

103. A method according to claim 101 wherein the phosphorylation is decreased by mutating the tyrosine and/or serine.

104. A method use according to claim 103 wherein the motif is mutated by substituting tyrosine for phenylalanine and/or substituting serine for glycine.

105. A method according to claim 101 wherein the phosphorylation is decreased by subjecting the cell to an antagonist which inhibits phosphorylation of the tyrosine and/or serine.

106. A method according to claim 101 wherein the cytokine mediated condition is a GM-CSF mediated condition.

107. A method according to claim 101 wherein the cytokine mediated condition involves cell survival.

108. A method according to claim 101 wherein the cytokine mediated condition involves cell proliferation.

109. A method according to claim 101 wherein the cytokine mediated condition is selected from the group consisting of myeloid cell activation, asthma and rheumatoid arthritis.

110. A method for diagnosing a proliferative condition involving cell proliferation or cell survival, said method including:
detecting a level of phosphorylation of tyrosine and/or serine in a bidentate motif capable of binding to a cytoplasmic protein comprising a tyrosine and a serine/threonine residue, said motif comprising an amino acid sequence alignment selected from the group consisting of:
N-X-X-Y-(X)₁₋₁₃-[R/K/H/Q]-[X/Ψ]₂₋₃-S/T-X-P (SEQ ID NO: 71)
wherein X is any residue, Y is tyrosine, S/T is serine or threonine and Ψ is a hydrophobic residue or an equivalent thereof; or
Y-(X)₁₋₁₆-[R/K/H/Q]-[X/Ψ]₂₋₃-S/T-X-P (SEQ ID NO: 72)
wherein X is any residue, Y is tyrosine, S/T is serine or threonine and Ψ is a hydrophobic residue or an equivalent thereof; or
N-X-X-Y-[X]₁₋₃₀-[R/K/Q/H]-[X]₁₋₄-[S/T]-X-p (SEQ ID NO: 73)
wherein X is any residue, Y is phosphotyrosine, S/T is phosphoserine/phosphothreonine; and comparing against a cell of a normal level of phosphorylation.
